(19) United States
(12) Patent Application Publication
(10) Pub. No.: US 2018/0250251 A1
(43) Pub. Date: Sep. 6, 2018

(54) Treatment of Patients with Familial Hypercholesterolemia on Lipid-Lowering Therapy

(71) Applicant: Gemphire Therapeutics Inc., Livonia, MI (US)

(72) Inventor: Charles L. Bisgaier, Ann Arbor, MI (US)

(21) Appl. No.: 15/956,232

(22) Filed: Apr. 18, 2018

Related U.S. Application Data
(63) Continuation-in-part of application No. 15/445,118, filed on Feb. 28, 2017.
Application No. 15/445,118 is a continuation of application No. PCT/US2017/019750, filed on Feb. 27, 2017.
(60) Provisional application No. 62/300,393, filed on Feb. 26, 2016.
(60) Provisional application No. 62/560,540, filed on Sep. 19, 2017.
(60) Provisional application No. 62/560,888, filed on Sep. 20, 2017.

(51) Int. Cl. A61K 31/194; A61K 45/06; A61P 3/06

(52) U.S. Cl. CPC A61K 31/194; A61P 3/06; A61K 45/06

(57) ABSTRACT

Methods for the treatment of homozygous familial hypercholesterolemia, compound heterozygous familial hypercholesterolemia, or heterozygous familial hypercholesterolemia by administering gemcabene as an adjunct to other lipid lowering therapy and/or modified diet.

### CROSS REFERENCE TO RELATED APPLICATIONS

This application claims the benefit of priority to U.S. Provisional Application No. 62/560,540 filed Sep. 19, 2017, and U.S. Provisional Application No. 62/560,888 filed Sep. 20, 2017, the entire contents of the aforementioned applications are hereby incorporated by reference herein.

### FIELD

Treatment of hypercholesterolemia, specifically patients with familial hypercholesterolemia (FH), including homozygous familial hypercholesterolemia (HoFH) and heterozygous familial hypercholesterolemia (HeFH).

### BACKGROUND

Familial hypercholesterolemia (FH) is a rare genetic disease that results when an individual inherits a substantial defect in clearance of low-density lipoprotein (LDL), resulting in dangerously high levels of circulating LDL cholesterol. Untreated it can cause premature coronary artery disease and stroke. FH is especially severe when the gene for FH is inherited from both parents, instead of just one, a condition referred to as homozygous familial hypercholesterolemia (HoFH).

HoFH is characterized by defective or deficient LDL receptors. HoFH can result from negative/deficient (<2% of normal LDL receptor activity) or defective (<30% of normal LDL receptor activity) LDL receptor activity. HoFH with negative receptors have much higher levels of LDL-C than those having at least one defective receptor. Untreated HoFH individuals have LDL-C levels that exceed 500 mg/dL (12.92 mmol/L) prior to treatment, cutaneous and tendinous xanthomata, corneal arcus and premature coronary artery disease. Untreated patients usually do not live past 20 to 30 years of age.

Besides mutations in the LDL receptor, at least three other genetic mutations can cause markedly elevated levels of plasma LDL-C and the cardiovascular consequences. First mutations in apolipoprotein B, can reduce or eliminate its ability to recognize and bind the LDL receptor, a condition known as “familial defective apolipoprotein B”. Second, conditions resulting in markedly elevated levels of PCSK9, can lead to rapid degradation of the LDL receptor, and result in elevated LDL-C levels. Third, mutations in the LDL-receptor adaptor protein 1 (LDL-RAP1) also impede clearance of LDL-C and leads to elevation of plasma LDL-C. Some individuals are refered to as compound heterozygous when they have two different mutations. Some individuals are referred to as HeFH when they have a single mutation that results in high LDL-C levels. LDL-C levels above 190 mg/dL are considered diagnostic for HeFH in the absence of lipid modifying agents. LDL-C levels above 190 mg/dL are also considered a clinical diagnostic for HeFH in the absence of genetic diagnosis.

The LDL-receptor mutations have developed in founder populations, in geographically isolated areas of the world. Generally, within these geographical locations the same allelic mutation penetrates the local gene pool, and the presentation of the mutation on one of the two gene alleles, is carried resulting in a heterozygous condition for the mutation. If both alleles of the gene are affected with the same mutation, a homozygous condition results.

Over time, as geographically isolated populations spread, genetic diversity allowed introduction of multiple mutations on the same gene. For the LDL-receptor, mutations in different locations on the gene allowed various differences in LDL receptor function. For example, there were founder populations that developed mutation in the ability of LDL to bind the LDL-receptor, to interalize the LDL-receptor (endocytosis), to effectively allow intracellular degradation of LDL, and to recycle the LDL receptor to the plasma membrane for efficient reutilization.

The genetic diversity allowed inheritance of separate mutations of the LDL receptor in the same individual, resulting in the condition still falling under the clinical category “Homozygous Familial Hypercholesterolemia”, but with the genetic designation of being “Compound Homozygous” for the LDL receptor. Both of the LDL receptor alleles are mutated, but at different loci.

Current treatment generally include a combination of dietary intervention, lipid regulating medications, and plasmapheresis or LDL apheresis. Current FDA approved treatments for HoFH include lipid-lowering therapy (lomitapide and mipomersen). In addition, statins, PCSK9 inhibitors, ezetimibe, bile acid sequestrants, LDL-apheresis, and liver transplant are also used for treatment. However, currently approved drugs have limitations in their effectiveness or have safety risks. For example, the product box labels for each of Lomitapide and Mipomersen includes as a Box Warning that they carry a Risk of Hepatotoxicity. In addition, even when combinations of therapies are utilized, the vast majority of patients still do not reach LDL-C levels considered optimal or consistent with halting the progression of coronary heart disease.

There is clearly a need for additional treatments that may provide an alternative to, or a combination with, the current treatments for Familial Hypercholesterolemia.

### SUMMARY

The present invention described herein provides an alternative to current treatment of HoFH and HeFH and may further be combined with current treatments. The present application discloses methods for treating patients with homozygous familial hypercholesterolemia (HoFH). The present application discloses methods for treating compound heterozygous patients. The present application discloses methods for treating patients with heterozygous familial hypercholesterolemia (HeFH). One embodiment of the present invention is a method for treating a patient with homozygous familial hypercholesterolemia, the method comprising administering an effective dose of gemcabene to the patient with homozygous familial hypercholesterolemia, wherein the patient is on a lipid-lowering therapy and is in need of further LDL-C lowering. Another embodiment is a method for reducing LDL-C in a patient with homozygous familial hypercholesterolemia, the method comprising administering an effective dose of gemcabene to the patient with homozygous familial hypercholesterolemia, wherein the patient is on a lipid-lowering therapy and the patient is in need of further LDL-C lowering. One embodiment of the present invention is a method for treating a patient with combined heterozygous familial hypercholesterolemia, the method comprising administering an effective dose of gemcabene to the patient with combined heterozygous familial hypercholesterolemia, wherein the patient is on a lipid-lowering therapy and is in need of further LDL-C lowering. Another embodiment is a method for reducing LDL-C in a patient with combined heterozygous familial hypercholesterolemia, the method comprising administering an effective dose of gemcabene to the patient with combined heterozygous familial hypercholesterolemia, wherein the patient is on a lipid-lowering therapy and the patient is in need of further LDL-C lowering. One embodiment of the present invention is a method for treating a patient with heterozygous familial hypercholesterolemia, the method comprising administering an effective dose of gemcabene to the patient with heterozygous familial hypercholesterolemia, wherein the patient is on a lipid-lowering therapy and is in need of further LDL-C lowering. Another embodiment is a method for reducing LDL-C in a patient with heterozygous familial hypercholesterolemia, the method comprising administering an effective dose of gemcabene to the patient with heterozygous familial hypercholesterolemia, wherein the patient is on a lipid-lowering therapy and the patient is in need of further LDL-C lowering.

### BRIEF DESCRIPTION OF THE FIGURES

FIGS. 1A, 1B and 1C provide a tabular listing of the Schedule of Procedures. FIG. 1A provides a summary for the screening procedure and the procedures during treatment of patients with 300 mg gemcabene. FIG. 1B provides a summary of the procedures during treatment patients with 600 mg gemcabene. FIG. 1C provides a summary of the procedures during treatment of patients with 900 mg gemcabene and during follow-up.

FIG. 2 is a graphical representation of the percent reduction in LDL-C in two HoFH patients after treatment with 300 mg and 600 mg of gemcabene.

FIG. 3 is a graphical representation of the mean percent change in LDL-C from baseline at 300 mg gemcabene treatment for 4 weeks (300 mg-Week 4), at 600 mg gemcabene for an additional 4 weeks (600 mg-Week 8), and at 900 mg gemcabene for an additional 4 weeks (900 mg-Week 12), in the overall study population (n=8), those patients genetically HoFH (n=3), and those patients genetically HeFH (n=5).

FIG. 4A is a graphical representation of percent change in LDL-C from pretreatment (baseline) over the 12 week gemcabene treatment period of all the individual patients and combined patients in the study. The treatment period consisted of a 12 week continuous treatment of a once oral daily gemcabene dose of 300 mg for 4 weeks, followed by a once oral daily gemcabene dose of 600 mg for 4 weeks, and then by a once oral daily gemcabene dose of 900 mg for 4 weeks.

FIG. 4B is a graphical representation of percent change in LDL-C from pretreatment (baseline) over the 12 week gemcabene treatment period of the individual HoFH patients and combined HoFH patients in the study. The treatment period consisted of a 12 week continuous treatment of a once oral daily gemcabene dose of 300 mg for 4 weeks, followed by a once oral daily gemcabene dose of 600 mg for 4 weeks, and then by a once oral daily gemcabene dose of 900 mg for 4 weeks.

FIG. 4C is a graphical representation of percent change in LDL-C from pretreatment (baseline) over the 12 week gemcabene treatment period of the individual HeFH patients and combined HeFH patients in the study. The treatment period consisted of a 12 week continuous treatment of a once oral daily gemcabene dose of 300 mg for 4 weeks, followed by a once oral daily gemcabene dose of 600 mg for 4 weeks, and then by a once oral daily gemcabene dose of 900 mg for 4 weeks.

FIG. 5 is a graphical representation of mean percent change in LDL-C from baseline over the 12 week gemcabene treatment period of the overall study population (n=8), EAS defined patients (n=6), the LDL receptor null patients (n=3) and the HeFH patients (n=5) in the study. The treatment period consisted of a 12 week continuous treatment of a once oral daily gemcabene dose of 300 mg for 4 weeks, followed by a once oral daily gemcabene dose of 600 mg for 4 weeks, and then by a once oral daily gemcabene dose of 900 mg for 4 weeks.

FIG. 6A is a graphical representation of mean percent change in LDL-C, ApoB, ApoE, and non-HDL-C from baseline over the 12 week gemcabene treatment period of the overall study population (n=8). The treatment period consisted of a 12 week continuous treatment of a once oral daily gemcabene dose of 300 mg for 4 weeks, followed by a once oral daily gemcabene dose of 600 mg for 4 weeks, and then by a once oral daily gemcabene dose of 900 mg for 4 weeks.

FIG. 6B is a graphical representation of mean percent change in LDL-C, ApoB, ApoE, and non-HDL-C from baseline over the 12 week gemcabene treatment period in the HoFH patients (n=3). The treatment period consisted of a 12 week continuous treatment of a once oral daily gemcabene dose of 300 mg for 4 weeks, followed by a once oral daily gemcabene dose of 600 mg for 4 weeks, and then by a once oral daily gemcabene dose of 900 mg for 4 weeks.

FIG. 6C is a graphical representation of mean percent change in LDL-C, ApoB, ApoE, and non-HDL-C from baseline over the 12 week gemcabene treatment period in the HeFH patients (n=5). The treatment period consisted of a 12 week continuous treatment of a once oral daily gemcabene dose of 300 mg for 4 weeks, followed by a once oral daily gemcabene dose of 600 mg for 4 weeks, and then by a once oral daily gemcabene dose of 900 mg for 4 weeks.

### DETAILED DESCRIPTION

Gemcabene provides an alternative to the current treatments for familial hypercholesterolemia without the safety concerns seen with prior drug treatments, such as increased risk of hepatotoxicity, or the invasiveness of LDL-apheresis or liver transplant. 6-(5-Carboxy-5-methyl-hexyloxy)-2,2-dimethyl-hexanoic acid, an ester, a pharmaceutically acceptable salt, or a hydrate thereof, may be used in the present invention. 6-(5-Carboxy-5-methyl-hexyloxy)-2,2-dimethyl-hexanoic acid is also known as gemcabene.

Gemcabene calcium is the monocalcium salt of a dialkyl ether dicarboxylic acid having 2 terminal gem dimethyl carboxylate moieties having the structure shown below:

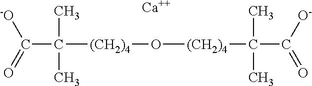

Gemcabene is a novel lipid-regulating compound with a multiple mechanism of action that involves: (1) blocking the hepatic production of triglyceride (TG); 2) blocking cholesterol synthesis; and (3) enhancing the clearance of very low-density lipoprotein. Based on prior clinical studies, the combined effects for these mechanisms has been observed to result in a reduction of plasma very low-density lipoprotein cholesterol (VLDL-C), low-density lipoprotein cholesterol (LDL-C), TG, as well as an elevation in high-density lipoprotein cholesterol (HDL-C). Gemcabene has also been shown to markedly lower C-reactive protein.

As noted in the background section conditions by which there is heterozygous inheritance of two or more separate mutant genes for any of the four genes associated with HoFH, could result in condition known as compound heterozygous and could lead to clinical defined HoFH. For example, a subject heterozygous for a LDL receptor mutation and heterozygous for an apoB mutation is genetically classified as a compound heterozygous, but may clinical present as a homozygous familial hypercholesterolemic subject. If only one allele is mutated that subject is considered to have heterozygous familial hypercholesterolemia (HeFH)

The examples in Table 1 show two separate alleic mutations at positions in the LDL-R gene, and one alleic mutation in the apoB gene. Mutations in the PCSK9 gene or the LDLRAP1 gene can also lead to genetic inheritance of familial hypercholesterolemia. As used in Table 1, position 1 refers to allelic position 1 and position 2 refers to allelic position 2.

TABLE 1
Examples of Genetic Inheritance and Terminology of Familial Hypercholesterolemia

| | | Genes Inherited from Mother | | | | |
|---|---|---|---|---|---|---|
| | | | | | | LDL-R |
| | | | | | | (Position 1) |
| | | | LDL-R | LDL-R | ApoB | plus ApoB |
| | Mutation | None | (Position 1) | (Position 2) | (Position 1) | (Position 1) |
| Genes Inherited | None | Normal | Heterozygous | Heterozygous | Heterozygous | Compound |
| from Father | | | | | | Heterozygous |
| | LDL-R | Heterozygous | Homozygous | Compound | Compound | Homozygous |
| | (Position 1) | | | Homozygous | Heterozygous | |
| | LDL-R | Heterozygous | Compound | Homozygous | Compound | Compound |
| | (Position 2) | | Homozygous | | Heterozygous | Homozygous |
| | ApoB | Heterozygous | Compound | Compound | Homozygous | Homozygous |
| | (Position 1) | | Heterozygous | Heterozygous | | |
| | LDL-R | Compound | Homozygous | Compound | Homozygous | Double |
| | (Position 1) | Heterozygous | | Homozygous | | Homozygous |
| | plus ApoB | | | | | |
| | (Position 1) | | | | | |

Genotype analysis for each of the four genes is not commonly conducted as the analysis is lengthy, expensive and interpretations of results controversial. For example polymorphic changes in DNA that result in a single amino acid or small changes may result in little or no functional change in the protein, but this genetic variation is considered a “mutation” or “variant” of the predominant gene in the population. The loose interpretation of functional activity does not allow precision in genetic classification. Furthermore, other genetic and environmental factors result in phenotypic variation.

For the above reasons, in medical practice, the classification of familial hypercholesterolemia, and more specifically homozygous familial hypercholesterolemia, is generally based on a clinical interpretation. The clinical interpretation is sometimes supported by follow-up by gene sequence analysis for both alleles of the LDL-receptor, apolipoprotein B, PCSK9 and LDL-RAP1 for the subject patient and if feasible the parents, siblings and other relatives.

### Definitions

“Subject” or “Patient” are used interchangeably.

The term “treating” or other forms of the word such as “treatment”, or “treat” is used herein to mean administration of a compound to mitigate a disease or a disorder in a host and/or reduces, inhibits, or eliminates a particular characteristic or event associated with a disorder. The term treating, and other forms of word treating, include prophylactic and therapeutic treatment.

Throughout the description and claims of this specification the word “comprise” and other forms of the word, such as “comprising” and “comprises,” means including but not limited to, and is not intended to exclude, for example, other additives, components, integers, or steps.

As used herein, the singular forms “a”, “an”, and “the” include plural references unless the context clearly dictates otherwise.

“Between” as used herein is inclusive, e.g., “between 1 mg and 5000 mg” includes 1 mg and 5000 mg.

“About” when used in conjunction with a number includes the number itself, for example, “from about 1 mg to about 5000 mg” includes the range “from 1 mg to 5000 mg”.

“From” as used herein is inclusive, e.g., “from 1 mg to 5000 mg” includes 1 mg and 5000 mg.

As used in the claims and embodiments herein, “Effective dose of gemcabene” is defined as a dose that reduces a HoFH patient's LDL-C level from baseline.

As used in the claims and embodiments herein, “baseline” or “baseline level of LDL-C” means the LDL-C level of a patient as measured prior to administration of gemcabene.

As used in the claims and embodiments herein, “lipid lowering therapy” includes treatment of patients with lipid-lowing medications, excluding gemcabene. Lipid lowering therapy does not exclude non-drug treatment, e.g., diet modification or LDL-apheresis.

As used herein, a “stable dose” means that the patient has been on the same dose of lipid-lowering medication for a period of time in which the level of LDL-C lowering has stabilized in the blood serum or plasma prior to administration of gemcabene.

As used herein LDL-C>500 mg/dL means that the LDL-C plasma concentration>500 mg/dL. Other similar reference to levels of LDL-C are interpreted the same unless the context clearly indicates otherwise.

As used in the present application, “a patient with HoFH” or “an HoFH patient” or the like, is a patient determined to have HoFH by genetic confirmation or clinical diagnosis. A patient with HoFH has (1) a genetic confirmation of two mutant alleles at the LDL-receptor, apolipoprotein B, PCSK9, or the LDL-RAP1 gene locus. For example the patient may have paired or same (homozygous) or two unpaired or dissimilar (compound homozygous or compound heterozygous) mutations at alleles on the LDL-receptor, apolipoprotein B, PCSK9, or the LDL-RAP1 gene locus; or (2) is clinically determined to have HoFH if (a) the patient has an untreated LDL-C>500 mg/dL (12.92 mmol/L) or treated LDL-C≥300 mg/dL (7.76 mmol/L) together with either appearance of cutaneous or tendinous xanthoma before 10 years of age, or evidence of heterozygous familial hypercholesterolemia in both parents; or (b) LDL-C>300 mg/dL (7.76 mmol/L) on maximally tolerated lipid-lowering drug therapy. The HoFH phenotype is only indicative, and low LDL-C levels, especially in children or in treated patients, do not exclude HoFH as a diagnosis.

CFR is an abbreviation for Code of Federal Regulations.

CYP is an abbreviation for cytochrome P450.

EAS is an abbreviation for the European Atherosclerosis Society.

EDC is an abbreviation for Electronic data capture.

HDL-C is an abbreviation for high-density lipoprotein cholesterol

HoFH is an abbreviation for homozygous familial hypercholesterolemia.

HeFH is an abbreviation for heterozygous familial hypercholesterolemia.

HsCRP is an abbreviation for high-sensitivity C-reactive protein.

LDL is an abbreviation for low-density lipoprotein.

LDL-C is an abbreviation for low-density lipoprotein cholesterol.

Lp(a) is an abbreviation for lipoprotein (a)

MedDRA is an abbreviation for Medical Dictionary for Regulatory Affairs.

NCEP ATP-III is an abbreviation for National Cholesterol Education Program Adult Treatment Panel III.

Non-HDL-C is an abbreviation for non-high-density lipoprotein cholesterol

PCSK9 is an abbreviation for proprotein convertase subtilisin/kexin type 9

QD is an abbreviation for once daily

TC is an abbreviation for total cholesterol.

TG is an abbreviation for triglyceride

Tₘₐₓ is an abbreviation for time to maximum plasma concentration.

Risk/Benefit

Depending on residual LDL receptor activity, patients often demonstrate a significantly limited response to otherwise highly efficacious statin therapy. Medications that have been approved specifically for the treatment of HoFH include lomitapide and mipomersen. Unfortunately, clinical application of these therapies is limited, as both treatments carry a product label BOX WARNING for hepatotoxicity. In addition, patients are often unable to tolerate other side effects of these medications, including injection site reactions and flu-like symptoms associated with mipomersen and gastrointestinal discomfort associated with lomitapide.

Whereas compounds such as mipomersen and lomitapide act late in the process of VLDL assembly, gemcabene reduces the synthesis of lipids required for lipoprotein assembly earlier in the process. This allows the precursors of cholesterol and fatty acid synthesis to be utilized in other metabolic processes without causing subsequent accumulation of intracellular TGs or hepatic fat leading to hepatic steatosis. Because gemcabene lowers TG levels, gemcabene may be useful for reducing the hepatic TG increase caused by administration of mipomersen or lomitapide.

The recently Food and Drug Administration (FDA)-approved proprotein convertase subtilisin/kexin type 9 (PCSK9) inhibitor, evolocumab (Repatha™), has demonstrated substantial LDL-C-lowering ability, but because the mechanism of action is dependent upon the presence of LDL-C receptors with some residual function, the LDL-C-lowering effect is typically reduced in patients with HoFH compared to that seen in other populations.

### EMBODIMENTS

In some embodiments gemcabene is administered at a dose from about 25 mg to about 900 mg daily. In some embodiments the dose of gemcabene is 25 mg, 50 mg, 75 mg, 150 mg, 300 mg, 450 mg, 600 mg or 900 mg. In some embodiments the dose of gemcabene is 150 mg, 300 mg, 600 or 900 mg. In some embodiments the dose of gemcabene is 300 mg, 600 or 900 mg. In some embodiments the daily dose of gemcabene is 25 mg, 50 mg, 75 mg, 150 mg, 300 mg, 450 mg, 600 mg or 900 mg. In some embodiments the daily dose of gemcabene is 150 mg, 300 mg, 600 mg or 900 mg. In some embodiments the daily dose of gemcabene is 300 mg, 600 mg or 900 mg.

Gemcabene may be administered 1, 2, 3, or 4 times per day. Preferably the gemcabene is administered 1 or 2 times a day. More preferably gemcabene is administered 1 time per day. Gemcabene's chemical formula is 6-(5-Carboxy-5-methyl-hexyloxy)-2,2-dimethyl-hexanoic acid. Gemcabene can be administered as an ester, a pharmaceutically acceptable salt, a hydrate or a combination of any of these forms. Gemcabene is generally administered as the monocalcium salt which is also referred to as gemcabene calcium.

As used herein, LDL-C level refers to the blood level of LDL-C unless specifically noted otherwise.

One embodiment of the present invention is a method for treating a patient with homozygous familial hypercholesterolemia (HoFH), the method comprising administering an effective dose of gemcabene to the patient with HoFH, wherein the patient is on a lipid-lowering therapy and is in need of further LDL-C lowering.

Another embodiment is a method for reducing LDL-C in a patient with HoFH, the method comprising administering an effective dose of gemcabene to the patient with HoFH, wherein the patient is on a lipid-lowering therapy and the patient is in need of further LDL-C lowering.

Another embodiment is the use of gemcabene to treat a patient with HoFH where the patient is administered an effective dose of gemcabene wherein the patient is on a lipid-lowering therapy and is in need of further LDL-C lowering.

Another embodiment is the use of gemcabene for reducing LCL-C in a patient with HoFH comprising administering an effective dose of gemcabene to the patient with HoFH, wherein the patient is on a lipid-lowering therapy and the patient is in need of further LDL-C lowering.

One embodiment of the present invention is a method for treating a patient with compound heterozygous familial hypercholesterolemia (CHFH), the method comprising administering an effective dose of gemcabene to the patient with CHFH, wherein the patient is on a lipid-lowering therapy and is in need of further LDL-C lowering.

Another embodiment is a method for reducing LDL-C in a patient with CHFH, the method comprising administering an effective dose of gemcabene to the patient with CHFH, wherein the patient is on a lipid-lowering therapy and the patient is in need of further LDL-C lowering.

Another embodiment is the use of gemcabene to treat a patient with CHFH where the patient is administered an effective dose of gemcabene wherein the patient is on a lipid-lowering therapy and is in need of further LDL-C lowering.

Another embodiment is the use of gemcabene for reducing LCL-C in a patient with CHFH comprising administering an effective dose of gemcabene to the patient with CHFH, wherein the patient is on a lipid-lowering therapy and the patient is in need of further LDL-C lowering.

One embodiment of the present invention is a method for treating a patient with heterozygous familial hypercholesterolemia (HeFH), the method comprising administering an effective dose of gemcabene to the patient with HeFH, wherein the patient is on a lipid-lowering therapy and is in need of further LDL-C lowering.

Another embodiment is a method for reducing LDL-C in a patient with HeFH, the method comprising administering an effective dose of gemcabene to the patient with HeFH, wherein the patient is on a lipid-lowering therapy and the patient is in need of further LDL-C lowering.

Another embodiment is the use of gemcabene to treat a patient with HeFH where the patient is administered an effective dose of gemcabene wherein the patient is on a lipid-lowering therapy and is in need of further LDL-C lowering.

Another embodiment is the use of gemcabene for reducing LCL-C in a patient with HeFH comprising administering an effective dose of gemcabene to the patient with HeFH, wherein the patient is on a lipid-lowering therapy and the patient is in need of further LDL-C lowering.

Still another embodiment is the use of gemcabene in the manufacture of a medicament for reducing LDL-C in a patient with HoFH wherein the patient is on a lipid-lowering therapy and the patient is in need of further LDL-C lowering.

In one embodiment the patient is on a lipid-lowering therapy for at least one week prior to the administration of gemcabene. In another embodiment the patient is on a lipid-lowering therapy for at least two weeks prior to the administration of gemcabene. In yet another embodiment the patient is on a lipid-lowering therapy for at least three weeks prior to the administration of gemcabene. In still another embodiment the patient is on a lipid-lowering therapy for at least four weeks prior to the administration of gemcabene. In one embodiment the patient is on a stable dose of the lipid-lowering therapy for at least one week prior to the administration of gemcabene. In another embodiment the patient is on a stable dose of the lipid-lowering therapy for at least two weeks prior to the administration of gemcabene. In yet another embodiment the patient is on a stable dose of the lipid-lowering therapy for at least three weeks prior to the administration of gemcabene. In still another embodiment the patient is on a stable dose of the lipid-lowering therapy for at least four weeks prior to the administration of gemcabene.

In some embodiments the patient is clinically determined to have HoFH. In some embodiments the patient is clinically determined to have HoFH because the patient has an untreated LDL-C>500 mg/dL (12.92 mmol/L) together with either appearance of cutaneous or tendinous xanthoma before 10 years of age, or evidence of heterozygous familial hypercholesterolemia in both parents. In another embodiment the patient is clinically determined to have HoFH because the patient has a treated LDL-C≥300 mg/dL (7.76 mmol/L) together with either the appearance of cutaneous or tendinous xanthoma before 10 years of age, or evidence of heterozygous familial hypercholesterolemia in both parents. In another embodiment the patient is clinically determined to have HoFH because the patient has an LDL-C>300 mg/dL (7.76 mmol/L) on maximally tolerated lipid-lowering drug therapy.

Untreated patients may present with an LDL-C level≥100 mg/dL, ≥130 mg/dL, ≥140 mg/dL, ≥150, ≥160 mg/dL, ≥170 mg/dL, ≥180 mg/dL, or ≥190 mg/dL. Some patients can have an LDL-C level≥400 mg/dL. Patients treated with a lipid lowering therapy may have LDL-C levels≥70 mg/dL, ≥90 mg/dL, ≥110 mg/dL, ≥200 mg/dL, or higher.

In other embodiments the patient is genetically confirmed as having HoFH. In one embodiment the genetically confirmed HoFH patient has a mutation in 2 alleles wherein the mutant alleles are mutations in the LDL-receptor gene, apolipoprotein B gene, PCSK9 gene or LDL-RAP gene. In some embodiments the patient with HoFH is determined to have paired or same (homozygous) or two unpaired or dissimilar (compound homozygous or compound heterozygous) mutations of alleles of the LDL-receptor, apolipoprotein B, PCSK9, or the LDL-RAP1 gene locus. In other embodiments the patient with Familial Hypercholesterolemia is determined to be heterozygous, homozygous, compound heterozygous, compound homozygus or double homozygous as set out in Table 1.

In some embodiments of the methods of the present invention, the patient's baseline LDL-C level is reduced by at least 15%. In other embodiments the patient's baseline LDL-C level is reduced by at least 20%. In still other embodiments the patient's baseline LDL-C level is reduced by at least 25%. In yet other embodiments the patient's baseline LDL-C level is reduced by at least 30%. In one embodiment the LDL-C level is reduced by at least 15% from baseline after four weeks of treatment with gemcabene. In another embodiment the LDL-C level is reduced by at least 15% from baseline after eight weeks of treatment with gemcabene. In still another embodiment the LDL-C level is reduced by at least 15% from baseline after twelve weeks of treatment with gemcabene.

In one embodiment of the disclosed methods the LDL-C level is reduced by at least 20% from baseline after four weeks of treatment with gemcabene. In another embodiment the LDL-C level is reduced by at least 20% from baseline after eight weeks of treatment with gemcabene. In still another embodiment the LDL-C level is reduced by at least 20% from baseline after twelve weeks of treatment with gemcabene.

In another embodiment of the disclosed methods the LDL-C level is reduced by at least 25% from baseline after four weeks of treatment with gemcabene. In another embodiment the LDL-C level is reduced by at least 25% from baseline after eight weeks of treatment with gemcabene. In still another embodiment the LDL-C level is reduced by at least 25% from baseline after twelve weeks of treatment with gemcabene.

In still another embodiment the disclosed methods the LDL-C level is reduced by at least 30% from baseline after four weeks of treatment with gemcabene. In another embodiment the LDL-C level is reduced by at least 30% from baseline after eight weeks of treatment with gemcabene. In still another embodiment the LDL-C level is reduced by at least 30% from baseline after twelve weeks of treatment with gemcabene.

Lipid-lowering therapies may comprise one or a combination of lipid lowering medicaments or lipid lowering diets. In some embodiments the lipid-lowering therapy comprises a statin. In some embodiments the statin is atorvastatin, or the statin is rosuvastatin, or the statin is simvastatin, or the statin is pravastatin, or the statin is lovastatin, or the statin is fluvastatin, or the statin is pitavastatin.

In other embodiments the lipid-lowering therapy comprises a cholesterol absorption inhibitor, an HMG-CoA reductase inhibitor, a PCSK9 inhibitor, an ACC inhibitor, an ApoC-III inhibitor, an Apo E mimetic, an Apo B synthesis inhibitor, a microsomal triglyceride transfer protein inhibitor, an ACL-inhibitor, fish oil, EPA, Lovaza, an ethyl ester of eicosapentaenoic acid, docosahexaenoic acid, an ethyl ester of docosahexaenoic acid, nicotinic acid, bile acid sequestrant, a CETP inhibitor or any combination thereof.

In one embodiment the lipid-lowering therapy comprises ezetimibe. In another embodiment the lipid-lowering therapy comprises mipomersen. In still another embodiment the lipid-lowering therapy comprises lomitapide. In yet another embodiment the lipid-lowering therapy comprises evolocumab.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

Prior Clinical Trials with Gemcabene

The safety and efficacy profile of gemcabene has been demonstrated through 18 Phase 1 and Phase 2 clinical studies (17 completed and 1 Phase 1 study stopped due to a business decision), involving 1272 healthy adult subjects and patients.

The clinical programs conducted to date has demonstrated that gemcabene is well tolerated. A total of 895 healthy adult subjects and patients with various underlying conditions (including dyslipidemia, osteoarthritis, and hypertension) have been exposed to a minimum of at least 1 dose of gemcabene at doses ranging from 25 mg to 1500 mg once daily (QD). This includes 837 subjects who received multiple doses of up to 900 mg daily for up to 12 weeks. Safety of these subjects was evaluated by regular adverse event monitoring, clinical laboratory assessments, electrocardiograms (ECGs), physical examinations, and vital sign assessments.

Phase 1 pharmacokinetic (PK) studies have demonstrated that gemcabene is rapidly absorbed following oral administration, with exposure increasing approximately linearly with dose. No significant drug-drug interactions have been observed with simvastatin (80 mg), atorvastatin (80 mg), or digoxin (0.25 mg). No clinically relevant effect on QTc interval or blood pressure has been observed.

Across all clinical studies, the majority of treatment-emergent adverse events were mild to moderate in intensity. The most common adverse events reported included headache, asthenia (feeling of weakness), nausea, dizziness, dyspepsia (upset stomach), infection, abnormal bowel movements, myalgia, and abnormal kidney function tests. Ten healthy adult patients reported a treatment-emergent serious adverse event (SAE) across all previous studies. None of these SAEs were considered treatment-related. There were no deaths.

Small mean increases in serum creatinine and blood urea nitrogen (BUN) have been observed in some studies. These changes appeared within the first 2 to 4 weeks and did not appear to increase further over time. An iohexol clearance study showed that glomerular filtration rate (GFR) slightly decreased and was associated with a slight increase in serum creatinine. There was no indication of proteinuria or hematuria identified in any subject. There were no significant changes observed in urine protein, suggesting that gemcabene does not cause tubular or glomerular injury. And, the increase was reversible with all creatinine values returning to baseline within approximately 2 weeks of cessation of gemcabene, suggesting a vascular effect and not renal injury.

COBALT-1 Trial

Described herein is a Phase 2 Open-Label, Dose-Finding Study to Assess the Efficacy, Safety, and Tolerability of Gemcabene in Patients with Homozygous Familial Hypercholesterolemia on Stable, Lipid-Lowering Therapy (COBALT-1).

The primary objective of this study is to evaluate the efficacy, safety, and tolerability of multiple doses of gemcabene in patients with HoFH on stable, lipid-lowering therapy.

The secondary objectives of this study are the following: to confirm the appropriate dose for use in Phase 3 registration studies as assessed by efficacy, pharmacokinetic (PK), and safety data (For the purposes of the trial, an effective dose is defined as a dose that achieves 15% mean reduction in LDL-C after 4 weeks of treatment); to further evaluate the efficacy of gemcabene in patients with HoFH following 4 weeks of dosing with gemcabene 300 mg once daily (QD), 4 weeks of dosing with gemcabene 600 mg QD, and 4 weeks of dosing with gemcabene 900 mg QD, as assessed by measurements of lipid and apolipoprotein parameters, high-sensitivity C-reactive protein (hsCRP), and fibrinogen; and to evaluate trough plasma concentrations of gemcabene at doses 300 mg, 600 mg, and 900 mg.

The exploratory objective of this study is to evaluate the effects of gemcabene on serum proprotein convertase subtilisin/kexin type 9 (PCSK9) levels.

Patient Population:

The population for this study is male and female patients, 17 years of age, diagnosed with HoFH by genetic confirmation or a clinical diagnosis based on either (1) a history of an untreated LDL-C concentration>500 mg/dL (12.92 mmol/L) together with either appearance of xanthoma before 10 years of age, or evidence of heterozygous familial hypercholesterolemia in both parents or, if history is unavailable, (2) LDL-C>300 mg/dL (7.76 mmol/L) on maximally tolerated lipid-lowering drug therapy. Patients must have a fasting LDL-C value>130 mg/dL (3.36 mmol/L) and a triglyceride (TG) value 400 mg/dL (4.52 mmol/L) at the Screening Visit while on a stable, low-fat, low-cholesterol diet in combination with a pre-existing, regulatory-approved, not excluded lipid-lowering therapy (i.e., statins, monoclonal antibodies to PCSK9, cholesterol-absorption inhibitors, bile acid sequestrants, or nicotinic acid, or any combination thereof.

Study Design and Duration:

This is a Phase 2, open-label, dose-finding, 3-period, 3-treatment study using successively escalating doses of 300 mg, 600 mg, and 900 mg gemcabene in patients with HoFH. All patients will be on each of the successive doses for 4 weeks at a time. Patients will remain on their current stable, lipid-lowering therapy throughout the study. Patients will not be allowed in the study if they are undergoing apheresis or taking mipomersen or lomitapide.

Efficacy, PK, and safety data from this study will be used along with previously completed studies and a planned randomized, placebo-controlled study in patients with hypercholesterolemia (GEM-301) to confirm the appropriate dose of gemcabene for use in Phase 3 studies.

Approximately 8 patients will be enrolled into the study. Total study duration will be up to 18 weeks and will consist of a Screening Visit, a Treatment Period, and a Follow-up Visit.

The Screening Visit will occur up to 14 days prior to Day 1. The Treatment Period is a sequential design whereby each patient will receive gemcabene 300 mg QD for 4 weeks. The same patients will then receive a 600 mg dose QD for 4 weeks and finally 900 mg dose QD for 4 weeks. There will be no interruptions in gemcabene dosing when changing from the 300 mg to the 600 mg dose or when changing from the 600 mg to the 900 mg dose unless there are clinically significant safety issues resulting in the temporary or permanent discontinuation of study drug. The first 300 mg dose of study drug will be administered at the site on Day 1. For days when patients will self-dose, they will be instructed to take study drug at the same time each morning on an empty stomach 30 to 60 minutes prior to breakfast. For patients also taking bile acid sequestrants, study drug should be taken at least 2 hours before administration of bile acid sequestrants. Assessments will be performed after the patient has been on the study drug for 2 weeks for each dosing level and on the last day of each dose.

For each escalated dose, percent change from baseline in LDL-C will be calculated using the baseline LDL-C value and the final LDL-C value measured for each dose. Baseline will be defined as the average of the Screening Visit occurring up to 14 days prior to Day 1 and Day 1 (pre-dose) measurements.

Pharmacokinetic samples will be collected pre-dose (must be 24±2 hours from the previous day's dose) and 0.5, 1, 2, 3, 5, and 12 hours post-dose on Day 28, Day 56, and Day 84 in collection tubes containing dipotassium ethylenediaminetetraacetic acid as the anticoagulant; for determination of gemcabene repeat-dose PK parameters, steady state is assumed following QD administration for 28 days and therefore, plasma gemcabene concentrations at 24 hours post-dose are considered to be equal to pre-dose concentrations. For all other study visits where routine plasma drug monitoring will be performed (Day 1, Day 14, Day 42, Day 70, and the Early Termination Visit, if applicable), samples will be collected pre-dose (must be 24±2 hours from the previous day's dose if a previous day's dose occurred).

The Follow-up Visit will occur 4 weeks (±3 days) after the last dose of study drug.

Dosage Forms and Route of Administration:

Study drug will be packaged in high-density polyethylene bottles with child-resistant closures. Patients will take the following for each of the 3 dose levels:

300 mg: one 300 mg tablet orally QD,

600 mg: two 300 mg tablets orally QD, and

900 mg: three 300 mg tablets orally QD.

Patients will be instructed to take study drug at the same time in the morning on an empty stomach 30 to 60 minutes prior to breakfast. For patients also taking bile acid sequestrants, study drug should be taken at least 2 hours before administration of bile acid sequestrants.

Efficacy Variables:

The primary efficacy analysis is the percent change in LDL-C from baseline to Day 28, Day 56, and Day 84.

The secondary efficacy analyses are the following:

The change in LDL-C from baseline to Day 28, Day 56, and Day 84;

The change and percent change in lipid parameters (non-high-density lipoprotein cholesterol [non-HDL-C], total cholesterol [TC], TG, high-density lipoprotein cholesterol [HDL-C], and very low-density lipoprotein cholesterol [VLDL-C]) from baseline to Day 28, Day 56, and Day 84;

The change and percent change in lipid parameters (non-HDL-C, TC, TG, HDL-C, and VLDL-C) from baseline to Day 28, Day 56, and Day 84 according to the receptor mutation status; the number (%) of patients achieving LDL-C reduction of ≥15%, ≥20%, ≥25%, and ≥30% at Day 28, Day 56, and Day 84; the number (%) of patients achieving an LDL-C value<100 mg/dL (2.59 mmol/L) at Day 28, Day 56, and Day 84, and at any time during the study; the change and percent change in apolipoprotein (Apo) B, ApoA-I, ApoA-II, ApoC-II, ApoC-III, ApoE, and lipoprotein(a) from baseline to Day 28, Day 56, and Day 84; the change and percent change in hsCRP from baseline to Day 28, Day 56, and Day 84; and the change and percent change in fibrinogen from baseline to Day 28, Day 56, and Day 84.

The exploratory analyses are the change and percent change in serum PCSK9 from baseline to Day 84.

Pharmacokinetic Variables:

The following PK parameters will be calculated, as appropriate, from the individual plasma concentrations of gemcabene on Day 28, Day 56, and Day 84: Cmax: maximum plasma concentration, tmax (h): time to maximum plasma concentration, AUC0-t (ng·h/mL): area under the concentration-time curve to the last quantifiable time, and AUC₀₋₂₄ (ng·h/mL): area under the concentration-time curve to the 24-hour time point.

Safety Variables:

The safety variables include adverse events; safety laboratory parameters (chemistry, hematology, coagulation, and urinalysis) with particular attention to hepatic (e.g., alanine aminotransferase/aspartate aminotransferase, bilirubin, alkaline phosphatase), renal (e.g., blood urea nitrogen, serum creatinine, protein:creatinine ratio, urinalysis sediments, pH, electrolytes), and skeletal muscle (i.e., creatine kinase) toxicities; 12-lead electrocardiograms (ECGs); physical examinations; and vital signs.

Statistical Analyses:

Given the proposed crossover design of this study, a within-patient analysis can be performed for the comparison of dose groups. For continuous variables, the dose groups will be compared on their change and percent reduction from baseline (using their pre-treatment baseline value). A longitudinal analysis will be performed with a mixed-effects model repeated measures analysis including percent change in LDL-C as the dependent variable, visit as a fixed effect and patient as a random effect. The additional drug benefit with increasing dose will be estimated from the mixed-effects model. Least-squares mean differences and corresponding 95% confidence intervals, separately for each of the 3 paired comparisons (300 versus 600 mg, 300 versus 900 mg, and 600 versus 900 mg) will be provided. In addition, a scatterplot with regression curve fit of the percent reduction from baseline versus dose will be performed. For binary variables such as the percentage of patients, descriptive statistics will be calculated for each dose group.

The PK parameters will be calculated, as appropriate, from the individual plasma concentrations of gemcabene using a non-compartmental approach. Pharmacokinetic variables will be computed using WinNonlin Professional® or other appropriate software. The following PK parameters will be calculated, as appropriate, from the individual plasma concentrations of gemcabene using a non-compartmental approach: Cₘₐₓ: maximum plasma concentration, tₘₐₓ (h): time to maximum plasma concentration, AUC₀₋ₜ (ng·h/mL): area under the concentration-time curve to the last quantifiable time, and AUC₀₋₂₄ (ng·h/mL): area under the concentration-time curve to the 24-hour time point.

Safety will be assessed using the population of all patients who receive any amount of study drug. The assessment of safety will include adverse events, clinical laboratory assessments, ECGs, physical examinations, and vital signs. The safety analysis will be based primarily on the frequency of new or worsening adverse events, laboratory abnormalities, and serious adverse events. Other safety data will be summarized as appropriate.

Safety laboratory data will be summarized at baseline, Day 28, Day 56, and Day 84, and change from baseline to Day 28, Day 56, and Day 84. Frequency counts of new or worsening abnormalities will also be provided.

Sample Size Determination:

The primary goal of the study is to assess the mean percent change in LDL-C from baseline over 12 weeks of treatment from the 3 dose levels. Dosing 8 patients per group will yield reasonable precision in estimation in mean change from baseline in LDL-C.

Study Objectives

Primary Objective

The primary objective of this study is to evaluate the efficacy, safety, and tolerability of multiple doses of gemcabene in patients with HoFH on stable, lipid-lowering therapy.

Secondary Objectives

The secondary objectives of this study are the following:

To confirm the appropriate dose for use in Phase 3 registration studies as assessed by efficacy, PK, and safety data (an effective dose is defined as a dose that achieves 15% mean reduction in LDL-C after 4 weeks of treatment);

To further evaluate the efficacy of gemcabene in patients with HoFH following 4 weeks of dosing with gemcabene 300 mg QD, 4 weeks of dosing with gemcabene 600 mg QD, and 4 weeks of dosing with gemcabene 900 mg QD, as assessed by measurements of lipid and apolipoprotein parameters, hsCRP, and fibrinogen; and

To evaluate trough plasma concentrations of gemcabene at doses 300 mg, 600 mg, and 900 mg.

Exploratory Objective

The exploratory objective of this study is to evaluate the effects of gemcabene on serum PCSK9 levels.

Study Description

Summary of Study Design

This is a Phase 2, open-label, dose-finding, 3-period, 3-treatment study using successively escalating doses of 300 mg, 600 mg, and 900 mg gemcabene in patients with HoFH. All patients will be on each of the successive doses for 4 weeks at a time. Patients will remain on their current stable, lipid-lowering therapy throughout the study. Patients will not be allowed in the study if they are undergoing apheresis or taking mipomersen or lomitapide.

Efficacy, PK, and safety data from this study will be used along with previously completed studies and a planned randomized, placebo-controlled study in patients with hypercholesterolemia (GEM-301) to confirm the appropriate dose of gemcabene for use in Phase 3 studies.

Approximately 8 patients will be enrolled into the study. Total study duration will be up to 18 weeks and will consist of a Screening Visit, a Treatment Period, and a Follow-up Visit.

The Screening Visit will occur up to 14 days prior to Day 1. Patients will sign the informed consent form (ICF) prior to any study procedures being performed. Patients must meet all of the inclusion and none of the exclusion criteria to be eligible for study participation.

The Treatment Period is a sequential design whereby each patient will receive gemcabene 300 mg QD for 4 weeks. The same patients will then receive a 600 mg dose QD for 4 weeks and finally 900 mg dose QD for 4 weeks. There will be no interruptions in gemcabene dosing when changing from the 300 mg to the 600 mg dose or when changing from the 600 mg to the 900 mg dose unless there are clinically significant safety issues resulting in the temporary or permanent discontinuation of study drug. The first 300 mg dose of study drug will be administered at the site on Day 1. For days when patients will self-dose, they will be instructed to take study drug at the same time each morning on an empty stomach 30 to 60 minutes prior to breakfast. For patients also taking bile acid sequestrants, study drug should be taken at least 2 hours before administration of bile acid sequestrants. Assessments will be performed after the patient has been on the study drug for 2 weeks for each dosing level and on the last day of each dose.

For each escalated dose, percent change from baseline in LDL-C will be calculated using the baseline LDL-C value and the final LDL-C value measured for each dose. Baseline will be defined as the average of the Screening Visit occurring up to 14 days prior to Day 1 and Day 1 (pre-dose) measurements.

Pharmacokinetic samples will be collected pre-dose (must be 24±2 hours from the previous day's dose) and 0.5, 1, 2, 3, 5, and 12 hours post-dose on Day 28, Day 56, and Day 84 in collection tubes containing dipotassium ethylenediaminetetraacetic acid (K₂EDTA) as the anticoagulant; for determination of gemcabene repeat-dose PK parameters, steady state is assumed following QD administration for 28 days and therefore, plasma gemcabene concentrations at 24 hours post-dose are considered to be equal to pre-dose concentrations. For all other study visits where routine plasma drug monitoring will be performed (Day 1, Day 14, Day 42, Day 70, and the Early Termination [ET] Visit, if applicable), samples will be collected pre-dose (must be 24±2 hours from the previous day's dose if a previous day's dose occurred)

The Follow-up Visit will occur 4 weeks (±3 days) after the last dose of study drug.

Study Indication: the indication for this study is for the treatment of hypercholesterolemia, specifically patients with HoFH.

Selection and Withdrawal of Patients

Inclusion Criteria

Patients who meet all of the following criteria will be eligible to participate in the study:

(1) Provision of written and signed informed consent (by patient or legal guardian) prior to any study-specific procedure; (2) Male or female≥17 years of age at time of consent; (3) Diagnosis of HoFH by genetic confirmation (including compound heterozygosity) or a clinical diagnosis based on either (a) a history of an untreated LDL-C concentration>500 mg/dL (12.92 mmol/L) together with either appearance of xanthoma before 10 years of age, or evidence of heterozygous familial hypercholesterolemia in both parents or, if history is unavailable, (b) LDL-C>300 mg/dL (7.76 mmol/L) on maximally tolerated lipid-lowering drug therapy; (4) Currently on a stable, low-fat, low-cholesterol diet in combination with a pre-existing, regulatory-approved, not excluded lipid-lowering therapy (i.e., statins, monoclonal antibodies to PCSK9, cholesterol-absorption inhibitors, bile acid sequestrants, or nicotinic acid, or any combination thereof) at a stable dose for at least 4 weeks prior to the Screening Visit; (5) Fasting LDL-C value>130 mg/dL (3.36 mmol/L) at the Screening Visit; (6) Physical examination, including vital signs, that is within normal limits or clinically acceptable to the Investigator; (7) Weight 50 kg; (8) Female patients must not be pregnant or lactating.

Exclusion Criteria

Patients who meet any of the following criteria will be excluded from participation in the study: Other forms of primary hyperlipoproteinemia and secondary causes of hypercholesterolemia (e.g., nephrotic syndrome or hypothyroidism); Abnormal liver function test at the Screening Visit (aspartate aminotransferase or alanine aminotransferase>2×the upper limit of normal [ULN]; total bilirubin>1.5×ULN; or alkaline phosphatase>2×ULN based on appropriate age and gender normal values). Patients with bilirubin>1.5×ULN and history of Gilbert's syndrome may be included; reflexive direct bilirubin testing will be used to confirm Gilbert's syndrome; Moderate (Grade B) or severe (Grade C) chronic hepatic impairment according to the Child-Pugh classification; Active liver disease (e.g., cirrhosis, alcoholic liver disease, hepatitis B virus [HBV], hepatitis C virus [HCV], autoimmune hepatitis, liver failure, liver cancer), history of liver transplant, or known diagnosis of human immunodeficiency virus (HIV); Triglycerides value>400 mg/dL (4.52 mmol/L) at the Screening Visit; Moderate to severe renal insufficiency defined as an estimated GFR<30 mL/min/1.73 m² (calculated using The Chronic Kidney Disease Epidemiology Collaboration equation) at the Screening Visit; Abnormal urinalysis (proteinuria greater than trace or any male or non-menstruating female with greater than trace hematuria), confirmed by reflexive urine protein:creatinine ratio testing; Uncontrolled thyroid disease: hyperthyroidism or hypothyroidism as defined by thyroid-stimulating hormone (TSH) below the lower limit of normal or >1.5×ULN, respectively, at the Screening Visit. If controlled, treatment should be stable for at least 3 months prior to the Screening Visit; Type 1 diabetes mellitus or uncontrolled type 2 diabetes mellitus (hemoglobin A1c [HbA1c] value>8%), or any diabetic patient taking insulin and/or thiazolidinediones; New York Heart Association Class III or IV heart failure; Myocardial infarction, severe or unstable angina pectoris, coronary angioplasty, coronary artery bypass graft, or other major cardiovascular events resulting in hospitalization within 3 months of the Screening Visit. Patients with adequately treated stable angina, per Investigator assessment, may be included; Uncontrolled cardiac arrhythmia or prolonged QT on the Screening Visit or Day 1 prior to dosing ECG (QTcF>450 msec for men and >470 msec for women) or known family history of prolonged QT or unexplained sudden cardiac death; Uncontrolled hypertension, defined as sitting systolic blood pressure>180 mmHg or diastolic blood pressure>110 mmHg, and confirmed by repeat measurement; Currently receiving cancer treatments or, in the Investigator's opinion, at risk of relapse for recent cancer; Use of fibrate lipid-lowering agent 6 weeks prior to the Screening Visit; Hypersensitivity to or a history of significant adverse reactions to any fibrate lipid-lowering agent; Use of apheresis (LDL or plasma) 8 weeks prior to the Screening Visit; Use of lomitapide 2 months prior to the Screening Visit; Use of mipomersen 5 months prior to the Screening Visit; Use of any excluded medications or supplements (e.g., potent cytochrome P450 [CYP] 3A4 inhibitors, see Appendix D); History of drug or alcohol abuse within the past year or inability to comply with protocol requirements, including subject restrictions (see Section [00191]); Previously treated with gemcabene; Participation in another clinical study of an investigational agent or device concurrently or within 1 month prior to the Screening Visit, or use of an investigational agent within 1 month or 5 half-lives (if known), whichever is longer, prior to the Screening Visit; or Any other finding which, in the opinion of the Investigator, would compromise the patient's safety or participation in the study.

Withdrawal Criteria

Participation of a patient in this clinical study may be discontinued for any of the following reasons: The patient withdraws consent or requests discontinuation from the study for any reason; Occurrence of any medical condition or circumstance that exposes the patient to substantial risk and/or does not allow the patient to adhere to the requirements of the protocol; Any SAE, clinically significant adverse event, severe laboratory abnormality, concomitant illness, or other medical condition which indicates to the Investigator that continued participation is not in the best interest of the patient; Pregnancy; Requirement of prohibited concomitant medication; Patient failure to comply with protocol requirements or study-related procedures; or Termination of the study by the Sponsor or the regulatory authority.

If a patient withdraws prematurely from the study due to the above criteria or any other reason, study staff should make every effort to complete the full panel of assessments scheduled for the ET Visit. The reason for patient withdrawal must be documented in the electronic Case Report Form (eCRF).

In the case of patients lost to follow-up, attempts to contact the patient must be made and documented in the patient's medical records.

Study Treatments

Treatment Groups

During the 12-week Treatment Period, all patients will receive gemcabene 300 mg QD for 4 weeks, followed by 600 mg QD for 4 weeks, followed by 900 mg QD for 4 weeks.

Rationale for Dosing

Based on the results from a completed clinical study (Study 1027-018), oral gemcabene significantly lowered LDL-C with mean percent changes of −23.4% and −27.7% at 300 mg and 900 mg, respectively, compared to −6.2% in the placebo group in hypercholesterolemic patients on stable statin therapy.

Gemcabene was observed to be well tolerated at single doses up to 1500 mg and multiple doses up to 900 mg. This included 837 subjects and patients with varying underlying conditions who received multiple doses of up to 900 mg for up to 12 weeks. Adverse events were generally mild to moderate in intensity with no treatment-related SAEs reported.

Randomization and Blinding

This is an open-label study, therefore, no randomization or blinding is necessary.

Breaking the Blind

This is an open-label study, therefore, no blinding is necessary.

Formulation and Packaging

The tablet drug product for oral administration is an immediate-release tablet containing 300 mg of the parent gemcabene in a formulation comprising the following inactive ingredients: lactose monohydrate, hydroxypropyl cellulose, croscarmellose sodium magnesium stearate, Opadry® White YS 1-7040, and Simethicone.

Study drug will be packaged in high-density polyethylene bottles with child-resistant closures. Patients will take the following for each of the 3 dose levels: 300 mg: one 300 mg tablet orally QD, 600 mg: two 300 mg tablets orally QD, and 900 mg: three 300 mg tablets orally QD.

Study Drug Preparation and Dispensing

Study drug will be administered at the site on days when study visits occur during the Treatment Period. Patients will self-dose at all other times during the Treatment Period. The Investigator or designee will provide patients with sufficient study drug until the next scheduled study visit.

Study Drug Administration

Patients will be instructed to take study drug at the same time in the morning on an empty stomach 30 to 60 minutes prior to breakfast. Missed doses will be documented. For patients also taking bile acid sequestrants, study drug should be taken at least 2 hours before administration of bile acid sequestrants. If a patient misses a dose, only a single dose (and not 2 doses) should be taken on the following day.

Treatment Compliance

Patients will be instructed to take study drug daily according to the protocol and return used and unused packaging to the site at each subsequent study visit. Compliance with administration of study drug will be assessed by means of tablet counts based on the assessment of empty bottles returned to the site at each study visit after Day 1 during the Treatment Period and the ET Visit, if applicable. Tablet counts will be recorded on the appropriate eCRF and the drug accountability log. The Investigator or designee will remind patients at each visit of the importance of following the protocol-defined schedule for taking study drug. Reasons for not following the study drug administration schedule as described in the protocol will be clearly recorded in the source documents.

Storage and Accountability

The study drug will be stored at room temperature (20±5° C.) in a secured location (locked) with access restricted to authorized personnel only. Storage temperature will be monitored and recorded. Upon receipt of study drug, the Investigator or designee will conduct a complete inventory of all study drug and ensure no damage occurred during shipment. The Investigator will maintain adequate records documenting the receipt, use, loss, or other disposition of study drug. Drug accountability logs will identify the study drug code number and account for the disposition on a patient-by-patient basis, including specific dates and quantities. The drug accountability logs will be signed by the individual who dispenses the study drug and copies will be provided to the Sponsor. All used and unused supplies will be appropriately inventoried and verified by the clinical research associate (CRA). Unused study drug may be destroyed at the sites according to their Standard Operating Procedures (SOPs). If a site does not have appropriate SOPs for compliance, the study drug will be returned to the Sponsor at the end of the study.

Prior and Concomitant Medications and/or Procedures

Patients are required to be on a stable, low-fat, low-cholesterol diet in combination with a pre-existing, regulatory-approved, not excluded lipid-lowering therapy (i.e., statins, monoclonal antibodies to PCSK9, cholesterol-absorption inhibitors, bile acid sequestrants, or nicotinic acid, or any combination thereof) during the study.

Patients are not permitted to receive treatment with lomitapide 2 months prior to the Screening Visit, mipomersen 5 months prior to the Screening Visit, or a fibrate lipid-lowering agent 6 weeks prior to the Screening Visit. Patients are not permitted to use strong CYP3A4 inhibitors while on the study drug.

Restrictions and Dietary Guidelines

It is important that patients are instructed to not undertake any form of strenuous physical activity for at least 24 hours prior to repeat blood testing. Patients are restricted from using alcohol within 48 hours prior to study visits. Assessments that require a patient to fast will be defined as no food or caloric beverage for at least 10 hours prior to sample collection. Patients will be permitted to have water. Study drug should be taken at the same time in the morning on an empty stomach 30 to 60 minutes prior to breakfast. For patients also taking bile acid sequestrants, study drug should be taken at least 2 hours before administration of bile acid sequestrants. Patients will be counseled on maintaining a low-fat, low-cholesterol diet (National Cholesterol Education Program Adult Treatment Panel III [NCEP ATP-III] or equivalent) throughout the study.

Documentation of Prior and Concomitant Medication Use

A concomitant medication is any treatment including nutritional supplements, vitamins, or over-the-counter medications received by or prescribed to the patient concomitantly to the study, from the time of informed consent to the Follow-up Visit or the ET Visit, if applicable. The Investigator should record the use of all concomitant medications taken during the study, both prescribed and over the counter, in the eCRF and the source document. This includes drugs used on a chronic and as needed basis. Patients should be discouraged from starting any new medication, both prescribed and over the counter, without consulting the Investigator, unless the new medication is required for an emergency.

Study Procedures

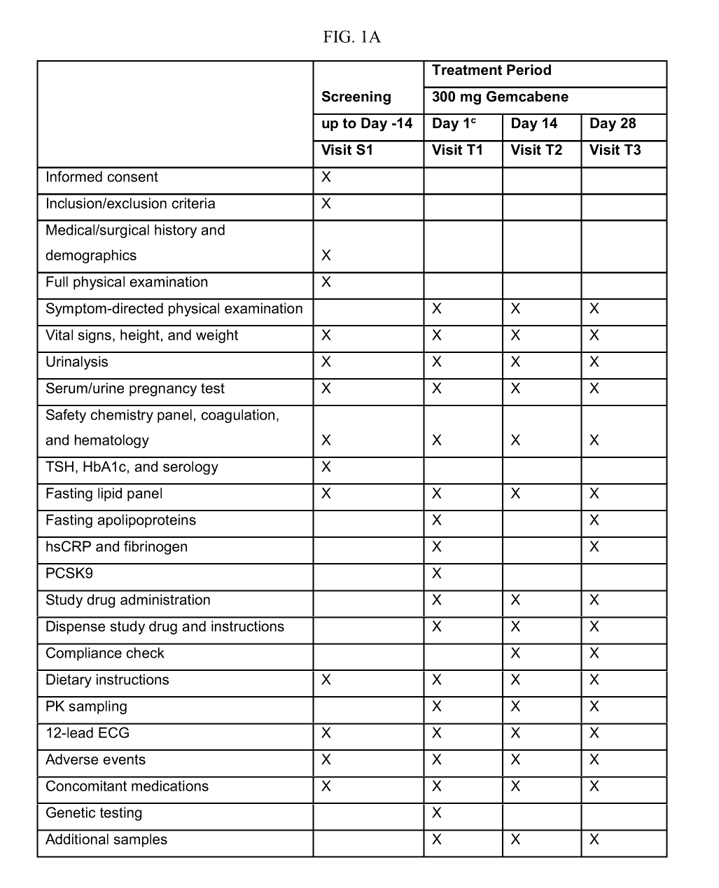

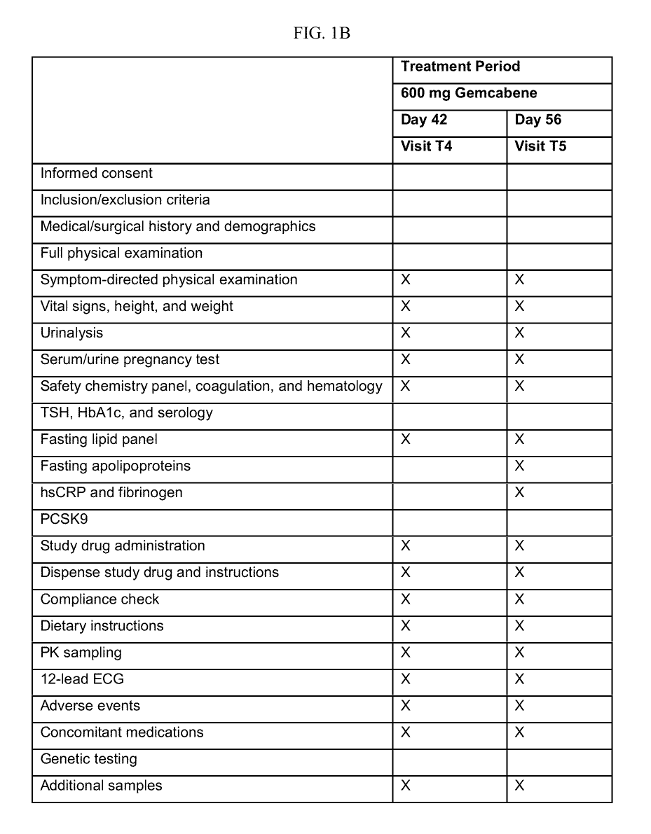

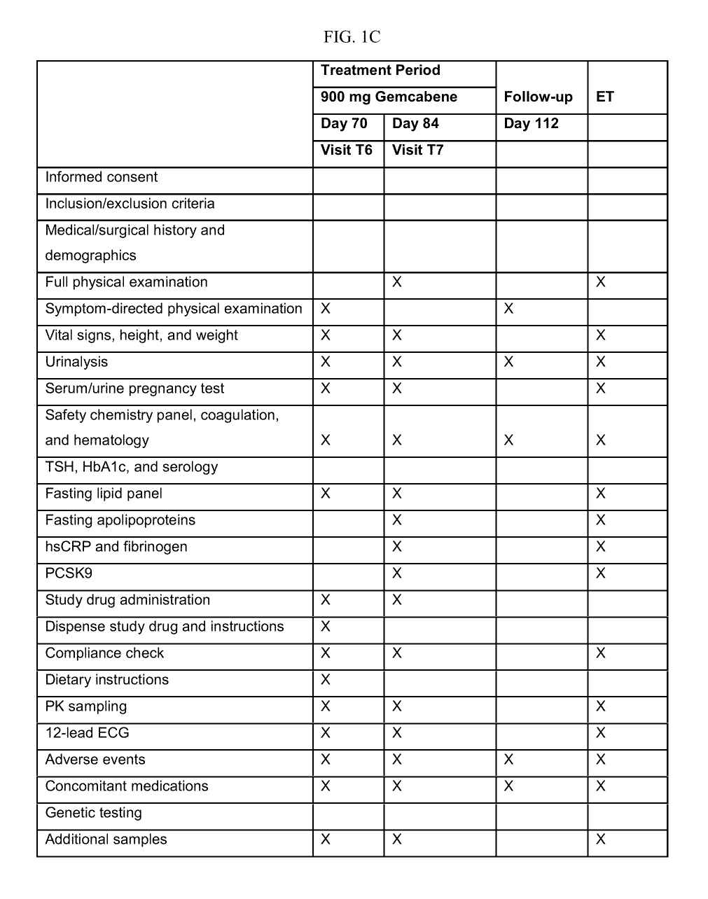

A tabular listing of the Schedule of Procedures can be found in FIGS. 1A, 1B, and 1C. Assessments that require a patient to fast will be defined as no food or caloric beverage for at least 10 hours prior to sample collection. Patients will be permitted to have water.

Efficacy Analyses

The following efficacy assessments will be measured in order to obtain the primary, secondary, and exploratory endpoints:

The following efficacy assessments will be measured in order to obtain the primary, secondary, and exploratory endpoints: Fasting ApoB, ApoA-I, ApoA-II, ApoC-II, ApoC-III, ApoE, and lipoprotein(a) (Lp[a]) at baseline, Day 28, Day 56, and Day 84 (or the ET Visit, if applicable); hsCRP at baseline, Day 28, Day 56, and Day 84 (or the ET Visit, if applicable); Fibrinogen at baseline, Day 28, Day 56, and Day 84 (or the ET Visit, if applicable); and Serum PCSK9 at baseline and Day 84 (or the ET Visit, if applicable).

Pharmacokinetic Assessments

The PK assessments of this study are to evaluate the gemcabene systemic exposure on Day 28, Day 56, and Day 84 and perform routine plasma drug monitoring on Day 1, Day 14, Day 42, and Day 70. Pharmacokinetic samples will be collected pre-dose (must be 24±2 hours from the previous day's dose) and 0.5, 1, 2, 3, 5, and 12 hours post-dose on Day 28, Day 56, and Day 84 in collection tubes containing K₂EDTA as the anticoagulant; for determination of gemcabene repeat-dose PK parameters, steady state is assumed following QD administration for 28 days and therefore, plasma gemcabene concentrations at 24 hours post-dose are considered to be equal to pre-dose concentrations. For all other study visits where routine plasma drug monitoring will be performed (Day 1, Day 14, Day 42, Day 70, and the ET Visit, if applicable), samples will be collected pre-dose (must be 24±2 hours from the previous day's dose if a previous day's dose occurred). The window for PK samples obtained at time intervals<24 hours will be ±10 minutes and the window for samples obtained at 24 hours will be ±2 hours.

Safety Assessments

An adverse event is defined as any untoward medical occurrence in a clinical investigation patient administered a pharmaceutical product, which does not necessarily have a causal relationship with this treatment. An adverse event can therefore be any unfavorable and/or unintended sign (including an abnormal laboratory finding), symptom, or disease temporally associated with the use of an investigational medicinal product, whether or not related to the investigational medicinal product. All adverse events, including observed or volunteered problems, complaints, or symptoms, are to be recorded on the appropriate eCRF.

Adverse events, which include abnormal and clinically significant clinical laboratory test variables, will be monitored and documented from the time of first dose of study drug (Day 1) until study participation is complete (the Follow-up Visit). Patients should be instructed to report any adverse event that they experience to the Investigator. Beginning with the signing of the informed consent until the time of the first dose of study drug (Day 1), investigators should make updates to medical history and record any pre-existing medical condition or signs or symptoms that changes in severity, frequency, or seriousness in the medical history. Serious adverse events that occur prior to the first dose of study drug (Day 1) should be reported as an update to medical history as well as be reported on the appropriate adverse event eCRF. Beginning with the first dose of study drug (Day 1), investigators should make an assessment for adverse events at each visit and record all adverse events, non-serious and serious, on the appropriate adverse event eCRF.

Wherever possible, a specific disease or syndrome rather than individual associated signs and symptoms should be identified by the Investigator and recorded on the eCRF. However, if an observed or reported sign or symptom is not considered a component of a specific disease or syndrome by the Investigator, it should be recorded as a separate adverse event on the eCRF. Additionally, the condition that led to a medical or surgical procedure (e.g., surgery, endoscopy, tooth extraction, or transfusion) should be recorded as an adverse event, not the procedure. Concomitant procedures should be recorded as such on the appropriate eCRF.

Any medical condition already present prior to the patient taking the first dose of study drug (Day 1) should be reported in the medical history. Any SAEs occurring prior to the first dose of study drug (Day 1) should be reported as an update to medical history as well as an adverse event. Any pre-existing medical condition or signs or symptoms that changes in severity, frequency, or seriousness after the patient takes the first dose of study drug (Day 1) and through the Follow-up Visit should be reported as an adverse event.

Clinically significant abnormal laboratory values or other examinations (e.g., ECG) that are detected at the time of the first dose of study drug (Day 1) and worsen during the study should be reported as adverse events. An abnormal laboratory result that is not verified by repeat testing does not necessitate reporting as an adverse event. The Investigator will exercise his or her medical, scientific, and clinical judgment in deciding whether an abnormal laboratory finding or other abnormal assessment is clinically significant. Clinically significant abnormal laboratory values occurring during the clinical study will be followed until repeat tests return to normal, stabilize, or are no longer clinically significant. Any abnormal test that is determined to be an error does not require reporting as an adverse event.

For adverse events with a causal relationship to study drug, follow-up by the Investigator will be required until the event or its sequelae resolve or stabilize to a level acceptable to the Investigator.

Unexpected Adverse Drug Reaction

An Unexpected Adverse Drug Reaction is defined as an adverse reaction, the nature or severity of which is not consistent with the applicable product information (see Investigator's Brochure). For gemcabene, the reference safety information is included in Sections 8.4 and 10 of the Investigator's Brochure currently in force. The reference safety information will be reviewed yearly and the periodicity of the review will be harmonized with the reporting period of the Development Safety Update Report.

Assessment of Adverse Events by the Investigator

The Investigator will assess the severity (intensity) of each adverse event as mild, moderate, or severe, and will also categorize each adverse event as to its potential relationship to study drug using the categories of Yes or No, as defined below.

Assessment of Severity: Mild—An event that is easily tolerated and generally not interfering with normal daily activities, Moderate—An event that is sufficiently discomforting to interfere with normal daily activities; Severe—An event that is incapacitating with inability to work or perform normal daily activities.

Causality Assessment. The relationship of an adverse event to the administration of the study drug is to be assessed according to the following definitions: No (unlikely related, unrelated, not related, no relation)—The time course between the administration of study drug and the occurrence or worsening of the adverse event rules out a causal relationship and another cause (e.g., medical history, concomitant drugs, therapies, and complications) is suspected. Yes (possibly related, related)—The time course between the administration of study drug and the occurrence or worsening of the adverse event is consistent with a causal relationship and no other cause (e.g., medical history, concomitant drugs, therapies, and complications) can be identified. The definition implies a reasonable possibility of a causal relationship between the event and the study drug. This means that there are facts (evidence) or arguments to suggest a causal relationship.

Specific Safety Measures

Hemoglobin Decrease

For a hemoglobin decrease of >1.5 g/dL from baseline during the study, repeat hematology studies and reflexive evaluation of reticulocyte count will be performed. The patient's past medical history, concomitant medications (including over the counter drugs and herbal supplements), and any recent symptoms (e.g., bleeding, shortness of breath, fatigue) will be reviewed to determine a potential etiology and make a clinical assessment of the significance of the finding.

Creatinine Increase

If, at any visit, a creatinine increase of >0.3 mg/dL (27 μmol/L) from baseline or a GFR decrease of >15 mL/min from baseline is observed, repeat chemistry will be performed. The patient's past medical history, concomitant medications (including over the counter drugs and herbal supplements), and any recent symptoms (e.g., fatigue, malaise, polyuria/oliguria, or palpitations) will be reviewed to determine a potential etiology and make a clinical assessment of the significance of the finding.

During the study, clinically significant abnormal results in NGAL will be used as a means of identifying patients who have unremarkable creatinine/BUN studies at the time of assessment but may require additional or closer/follow-up monitoring of renal studies.

Possible Muscle and Liver Injury

For muscle injury, creatine kinase (CK), hepatic, and renal function laboratory data will be integrated with myopathy signs and symptoms. For management of CK elevations>3×ULN, refer to Appendix E. For liver injury, laboratory data will be integrated with hepatic signs and symptoms. Alanine aminotransferase increases>2×ULN with symptoms of hepatitis or >3×ULN with or without symptoms of hepatitis will be evaluated and managed according to guidelines.

Serious Adverse Events

An adverse event or adverse reaction is considered serious if, in the view of either the Investigator or Sponsor, it results in any of the following outcomes: Death; A life-threatening adverse event; Requires hospitalization or prolongation of existing hospitalizations; A persistent or significant disability/incapacity or substantial disruption of the ability to conduct normal life functions; A congenital anomaly/birth defect; or An important medical event.

Follow-Up Reports

The Investigator must continue to follow the patient until the SAE has subsided or until the condition becomes chronic in nature, stabilizes (in the case of persistent impairment), or the patient dies.

Pregnancy Reporting

If a patient participating in the study becomes pregnant during the study or within 30 days of discontinuing study drug, the Investigator should report the pregnancy to the Clinical Safety Group within 24 hours of being notified.

Clinical Laboratory Evaluations

Clinical laboratory evaluations will be collected at the visits shown in the Schedule of Procedures (FIGS. 1A, 1B and 1C) and the data captured will be forwarded to the central laboratory for evaluation. Assessments that require a patient to fast will be defined as no food or caloric beverage for at least 10 hours prior to sample collection. Patients will be permitted to have water.

Standard clinical laboratory evaluations for safety chemistry, coagulation, and hematology will be conducted at all study visits and the Follow-up Visit (only for patients who had an abnormal result at Day 84 [or the ET Visit, if applicable] or an ongoing treatment-related adverse event). Clinically significant abnormal creatinine results at Day 84 (or the ET Visit, if applicable) will also be followed-up 2 weeks (±3 days) after the last dose of study drug in addition to the 4 week (±3 days) Follow-up Visit. A fasting lipid panel will be assessed at all study visits, excluding the Follow-up Visit. Fasting apolipoproteins, hsCRP, and fibrinogen will be assessed at Day 1, Day 28, Day 56, Day 84, and the ET Visit, if applicable. In addition to these lipid parameters, PCSK9 will also be measured at Day 1, Day 84, and the ET Visit, if applicable.

A urine sample for urinalysis will be collected at all study visits and the Follow-up Visit (only for patients who had an abnormal result at Day 84 [or the ET Visit, if applicable] or an ongoing treatment-related adverse event). A urine microscopic examination will be performed when the dipstick result is abnormal (positive for blood, leukocyte esterase, or nitrites). Urine protein:creatinine ratio will be performed at the Screening Visit, Day 1, Day 28, Day 56, Day 84, the Follow-up Visit (only for patients who had an abnormal result at Day 84 [or the ET Visit, if applicable] or an ongoing treatment-related adverse event), and the ET Visit, if applicable. Urinary NGAL will be measured at Day 1, Day 28, Day 56, Day 84, the Follow-up Visit (only for patients who had an abnormal result at Day 84 [or the ET Visit, if applicable] or an ongoing treatment-related adverse event), and the ET Visit, if applicable.

Serology tests for HBV, HCV, and HIV will be conducted at the Screening Visit. For women of child-bearing potential only, a serum pregnancy test will be conducted at the Screening Visit, Day 84, and the ET Visit, if applicable. A urine pregnancy test will be conducted at all other study visits, excluding the Follow-up Visit. Thyroid-stimulating hormone and HbA1c will be measured at the Screening Visit.

Measurement of vital signs will include an assessment of pulse rate, blood pressure, respiration rate, and temperature. Vital signs will be measured at all study visits, excluding the Follow-up Visit. Blood pressure should be obtained in the seated position, after the patient has rested comfortably for at least 5 minutes.

Electrocardiograms will be performed in triplicate and sent to a central reviewer. Patients should be lying quietly in a fully supine position for at least 10 minutes prior to each 12-lead ECG. A 12-lead ECG will be performed at the Screening Visit and pre-dose on Day 1, Day 14, Day 42, Day 70, and the ET Visit, if applicable. Electrocardiograms will be performed pre-dose and 2 hours post-dose on Day 28, Day 56, and Day 84. The Investigator will assess ECG data as normal, abnormal not clinically significant, or abnormal clinically significant. Any clinically significant abnormalities should be documented as medical history/adverse event/SAE as applicable. All ECG tracings will be kept as source data.

A full physical examination will be performed at the Screening Visit, Day 84, and the ET Visit, if applicable, and includes genitourinary examination per the Investigator's discretion and does not include a rectal examination. Assessment for xanthoma or arcus should also be part of the full physical examination.

A symptom-directed physical examination will be conducted at all other study visits and the Follow-up Visit (only for patients who had an abnormal result at Day 84 [or the ET Visit, if applicable] or an ongoing treatment-related adverse event).

Genetic Testing

Peripheral blood cell DNA for determination of genetic testing for the HoFH genotype mutational status will be collected at Day 1 for all patients. This data will be used to confirm diagnosis, categorize receptor function according to published data, and possibly show responses for receptor negative patients (if enrolled) separately from those with at least one defective receptor.

Additional Samples

Additional blood samples will be collected at all study visits during the Treatment Period and the ET Visit, if applicable, to be available for analysis of exploratory biomarkers associated with lipid metabolism, repeat lipid testing, blood drug levels, and/or repeat or additional clinical laboratory and urine testing in the event of a safety issue.

Missing Data

The primary analyses of the primary and secondary outcome variables will use linear mixed effects models. This analysis method will allow for inclusion of patients with missing values thus using the maximum amount of data for the analysis and making fewer assumptions about the missing data compared to a more traditional per protocol analysis.

To summarize laboratory variables, consecutive time windows will be created around each planned visit. In the descriptive statistics of laboratory variables, only measurements from scheduled visits will be used if values are available. If no values from a scheduled visit are available but values from unscheduled visits are available, the values from the last unscheduled visit from that window will be used for the summary statistics. The results of all laboratory values from unscheduled and repeat measurements will be recorded in the clinical database. In listings and narratives, all laboratory values including unscheduled and repeat values will be included.

Analysis of Safety

Safety will be assessed using the population of all patients who receive any amount of study drug. The assessment of safety will include adverse events, clinical laboratory assessments, ECGs, physical examinations, and vital signs. The safety analysis will be based primarily on the frequency of new or worsening adverse events, laboratory abnormalities, and SAEs. Other safety data will be summarized as appropriate.

Sample Size Determination

The primary goal of the study is to assess the mean percent change in LDL-C from baseline over 12 weeks of treatment from the 3 dose levels. Dosing 8 patients per group will yield reasonable precision in estimation in mean change from baseline in LDL-C.

Data Management

Data will be recorded at the site on eCRFs and reviewed by the CRA during monitoring visits. The CRAs will verify data recorded in the EDC system with source documents. All corrections or changes made to any study data must be appropriately tracked in an audit trail in the EDC system. An eCRF will be considered complete when all missing, incorrect, and/or inconsistent data has been accounted for.

Data will be collected and processed using a validated EDC system. The system and procedures are designed in compliance with Title 21 of the Code of Federal Regulations (21 CFR Part 11).

For medical information, the following thesauri will be used: Latest version of MedDRA for medical history and adverse events, and World Health Organization Drug Dictionary for prior and concomitant medications.

Validation checks programmed within the EDC system, as well as supplemental validation performed via review of the downloaded data, will be applied to the data in order to ensure accurate, consistent, and reliable data. Data identified as erroneous, or data that are missing, will be referred to the investigative site for resolution through data queries.

Investigator Requirements and Quality Control

Ethical Conduct of the Study. Good Clinical Practice (GCP) is an international ethical and scientific quality standard for designing, conducting, recording, and reporting studies that involve human patients. Compliance with this standard provides public assurance that the rights, safety, and well-being of study patients are protected, consistent with the principles that have their origin in the Declaration of Helsinki, and that the clinical study data are credible.

Institutional Review Board/Ethics Committee

Federal regulations and the International Conference on Harmonization (ICH) require that approval be obtained from an Institutional Review Board (IRB)/Ethics Committee (EC) prior to participation of patients in research studies. The IRB/EC will review all appropriate study documentation in order to safeguard the rights, safety, and well-being of patients. The study will only be conducted at sites where IRB/EC approval has been obtained. The protocol, Investigator's Brochure, ICF, advertisements (if applicable), written information given to the patients, safety updates, annual progress reports, and any revisions to these documents will be provided to the IRB/EC by the Investigator.

Study Monitoring Requirements

It is the responsibility of the Investigator to ensure that the study is conducted in accordance with the protocol, Declaration of Helsinki, ICH GCP, Directive 2001/20/EC, and applicable regulatory requirements (e.g., 21 CFR 312 Part D), and that valid data are entered into the eCRFs. The role of the study monitor is to verify the rights and well-being of the patients are protected, the data is accurate, complete, and verifiable from source documents, and the conduct of the study is in compliance with the protocol, Declaration of Helsinki, ICH GCP, and applicable regulatory requirements. To achieve this objective, the monitor's duties are to aid the Investigator and, at the same time, the Sponsor in the maintenance of complete, legible, well organized and easily retrievable data. Before the enrollment of any patient in this study, the Sponsor or their designee will review with the Investigator and site personnel the following documents: protocol, Investigator's Brochure, eCRFs and procedures for their completion, informed consent process, management of investigational product, and the procedure for reporting adverse events such as SAEs. All monitoring activities will be reported and archived. In addition, monitoring visits will be documented at the investigational site by signature and date on the study-specific monitoring log and findings documented in a follow-up letter.

Results

This application describes a clinical trial that produced surprising evidence of the significant ability of gemcabene to lower LDL-C levels in familial hypercholesterolemic patients. Gemcabene lowered total cholesterol, non-HDL, apoB, apoE and hsCRP levels. Gemcabene was safe and well tolerated in combination with standard of care lipid modifying therapies.

### EXAMPLES

### Example 1

Two HoFH male patients treated per the protocol described above showed reductions of greater than 15 percent in LDL-C beyond maximal lipid-lowering therapies. Both patients were determined to be compound heterozygous by genotyping.

Patient 1 was continued on treatment with 40 mg rosuvastatin once daily. After four weeks of treatment with 300 mg/day gemcabene, the patient's LDL-C level was reduced from the baseline level by 28.7%. The patient's dose was then increased to 600 mg/day, per the protocol, and after four weeks treatment the patient's LDL-C level was reduced from baseline by 32.4%. (see Table 2)

Patient 2 was maintained on 80 mg/day atorvastatin and 10 mg/day ezetimibe. After four weeks of treatment with 300 mg/day gemcabene, the patient's LDL-C level was reduced from the baseline level by 18.3%. The patient's dose was then increased to 600 mg/day, per the protocol, and after four weeks treatment the patient's LDL-C level was reduced from baseline by 22.9%. (See Table 2).

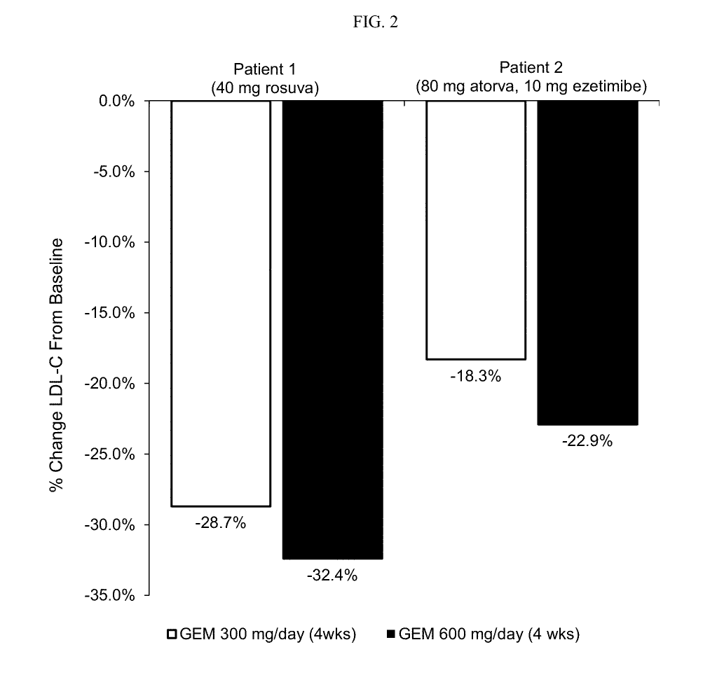

The results are graphically depicted in FIG. 2.

TABLE 2

| | | | | | % Change | % Change |
|---|---|---|---|---|---|---|
| | | | | | From | From |
| | | | Maximal | | Baseline, | Baseline, |
| | | | Lipid- | Baseline | Gemcabene | Gemcabene |
| | | HoFH Entry | Lowering | LDL-C | 300 mg/day | 600 mg/day |
| Patient | Gender | Criteria | Therapies | mg/dL | (4 weeks) | (4 weeks) |
| 1 | Male | Genotype | Rosuvastatin | 138 | −28.7% | −32.4% |
| | | (Compound | 40 mg | | | |
| | | Heterozygous) | | | | |
| 2 | Male | Genotype | Atorvastatin | 195 | −18.3% | −22.9% |
| | | (Compound | 80 mg | | | |
| | | Heterozygous) | Ezetimibe | | | |
| | | | 10 mg | | | |

### Example 2

The Patients in Example 1 were evaluated for an additional 4 weeks on 900 mg/day and an additional 6 patients were evaluated for the full 12 weeks. Table 3 shows the subject demographics.

TABLE 3

| | | | BL Criteria for Study | Study | |
|---|---|---|---|---|---|
| | | | Entry | Genetic | Concomitant Lipid |
| Age | Gender | Race | (Genetic/Clinical) | Assessment | Medications |
| 58 | Male | Caucasian | Genetic: compound | HeFH | Atorvastatin 80 mg; |
| | | | heterozygosity | | ezetimibe 10 mg |
| 71 | Female | Caucasian | Clinical | HeFH | Ezetimibe 10 mg; |
| | | | | | cholestyramine 4 g; |
| | | | | | krill oil 350 mg |
| 56 | Male | Caucasian | Genetic: compound | HeFH | Rosuvastatin 40 mg |
| | | | heterozygosity | | |
| 47 | Male | Caucasian | Genetic: HoFH | HoFH | Atorvastatin 80 mg, |
| 42 | Male | Caucasian | Genetic: HoFH | HoFH | Atorvastatin 80 mg, |
| | | | | | ezetimibe 10 mg, |
| | | | | | evolocumab 140 mg |
| 59 | Female | Caucasian | Genetic: HoFH | HeFH | Evolocumab 140 mg |
| 25 | Male | Caucasian | Genetic: HoFH | HoFH | Atorvastatin 80 mg; |
| | | | | | ezetimibe 10 mg; |
| | | | | | Evetol 100 mg |
| 65 | Female | Caucasian | Clinical | HeFH | Ezetimibe 10 mg |

The eight patients were genetically analyzed to evaluate the location of mutations. Table 4 below sets out their mutations.

TABLE 4

| | | | Met EAS Consensus | Overall |
|---|---|---|---|---|
| | | | Panel Clinical | Genetic |
| Age | LDL-R | Apo-B | Diagnosis of HoFH | Definition |
| 58 | ~2.6 kb CNV deletion | No mutation identified | Yes | HeFH |
| | spanning 5′UTR, exon1 | | | |
| | and intron 1 - | | | |
| | heterozygote | | | |
| 71 | No mutation identified | A73D heterozygote | No | HeFH |
| 56 | W87G heterozygote | No mutation identified | Yes | HeFH |
| 47 | Y828C homozygote | No mutation identified | Yes | HoFH |
| | | | | (Null LDLr) |
| 42 | Y828C homozygote | No mutation identified | Yes | HoFH |
| | | | | (Null LDLr) |
| 59 | C681X heterozygote | No mutation identified | Yes | HeFH |
| 25 | Y188X homozygote | No mutation identified | Yes | HoFH |
| | | | | (Null LDLr) |
| 65 | Intron 17 -14C > G | G753E heterozygote; | No | HeFH |
| | heterozygote | Y129C heterozygote | | |

Baseline Demographics are shown in Table 5

TABLE 5

| | Subjects (n = 8) |
|---|---|
| Age mean (years) | 53 |
| Caucasian | 8 (100%) |
| Male | 5 (63%) |
| Background lipid lowering treatment | 8 (100%) |
| Statin | 5 (63%) |
| Ezetimibe | 5 (63%) |
| Anti-PCSK9 | 2 (25%) |
| BMI (kg/m2) | 27.7 ± 6.3 |
| Baseline LDL-C (SD) | 351.3 ± 170.1 |

As noted above, all patients are required to be on a stable, low-fat, low-cholesterol diet in combination with a pre-existing, regulatory-approved, not excluded lipid-lowering therapy (i.e., statins, monoclonal antibodies to PCSK9, cholesterol-absorption inhibitors, bile acid sequestrants, or nicotinic acid, or any combination thereof) during the study. Table 3 above indicates which lipid lowering therapy drugs the patients were taking through the trial.

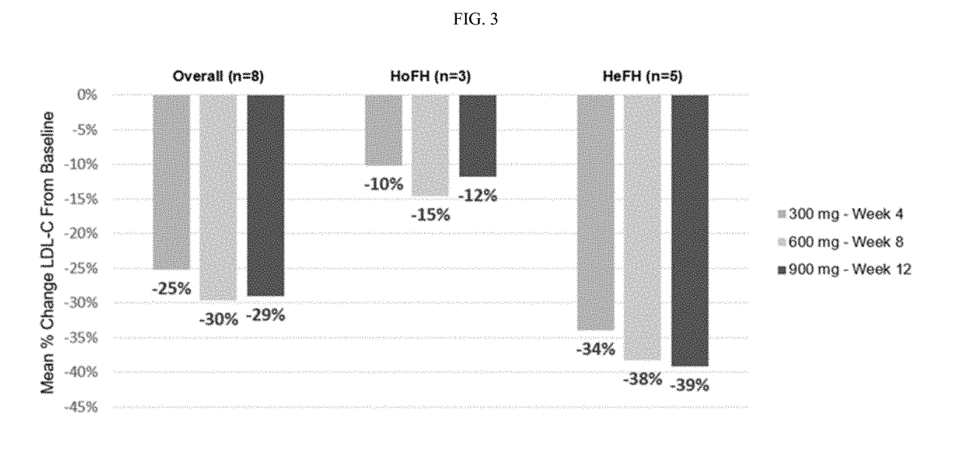

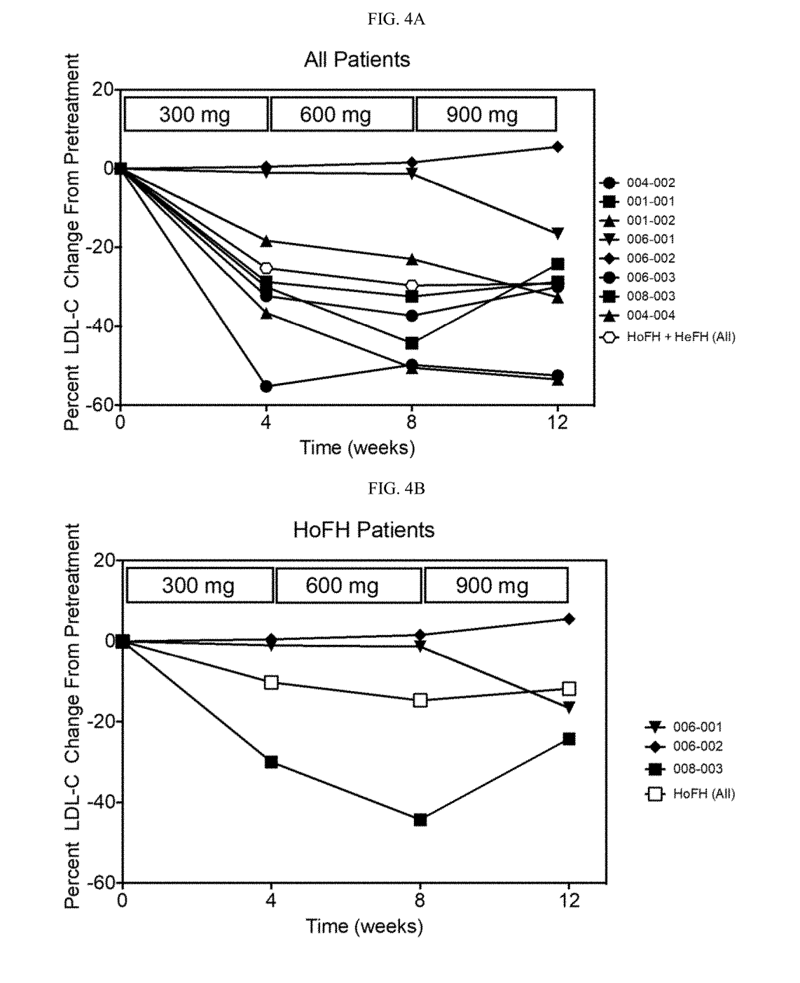

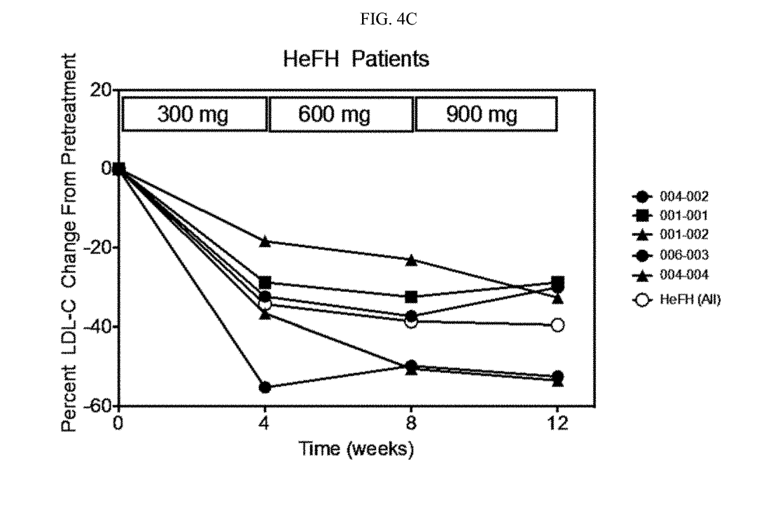

Patients were treated for 4 weeks with 300 mg/day, followed by 4 weeks with 600 mg/day, followed by 4 weeks of 900 mg/day of gemcabene. LDL-C levels prior to treatment and after each of the 4 week treatments was determined. LDL-C changes in LDL-C mg/dL levels by dose are shown in FIG. 3. FIG. 3 shows three graphs, the first is for the LDL-C levels for all patients at the three doses (all), the second graph shows the LDL-C levels for the HoFH patients, and the third graph shows the LDL-C levels for the HeFH patients. The LCL-C levels per patient separated into these three groups are shown in FIGS. 4A, 4B and 4C.

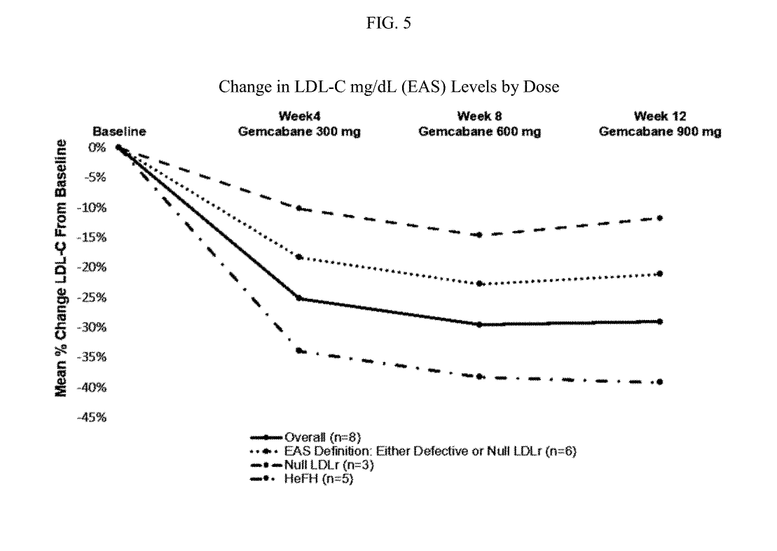

The numbers from this study were also divided by the European Atherosclerosis Society (EAS) calculation as shown in Table 6 below. The percent change from baseline data are presented in FIG. 5.

TABLE 6
EAS Change in LDL-C Levels by Dose

| | | Gemcabene 300 mg | | Gemcabene 600 mg | | Gemcabene 900 mg | |
|---|---|---|---|---|---|---|---|
| | | Week 4 | | Week 8 | | Week 12 | |
| | | | % | | % | | % |
| | | Change in | Change | Change in | Change | Change in | Change |
| | Baseline LDL- | LDL-C from | from | LDL-C from | from | LDL-C from | from |
| | C mg/dL (SD) | Baseline (SD) | Baseline | Baseline (SD) | Baseline | Baseline (SD) | Baseline |
| Overall | 351.3 (181.8) | −75.6 (67.8) | −25.2% | −93.3 (92.9) | −29.6% | −88.4 (62.8) | −29.1% |
| (n = 8) | | | | | | | |
| EAS | 374.3 (208.7) | −58.3 (70.0) | −18.3% | −77.2 (103.9) | −22.8% | −68.0 (58.8) | −21.1% |
| Definition: | | | | | | | |
| Either | | | | | | | |
| Defective | | | | | | | |
| or Null | | | | | | | |
| LDLr (n = 6) | | | | | | | |
| Null LDLr | 551.3 (105.7) | −63.7 (106.9) | −10.2% | −92.3 (159.2) | −14.7% | −75.7 (90.0) | −11.8% |
| (n = 3) | | | | | | | |
| HeFH | 231.2 (65.2) | −82.8 (46.5) | −34.2% | −93.8 (49.2) | −38.6% | −96.0 (51.5) | −39.5% |
| (n = 5) | | | | | | | |

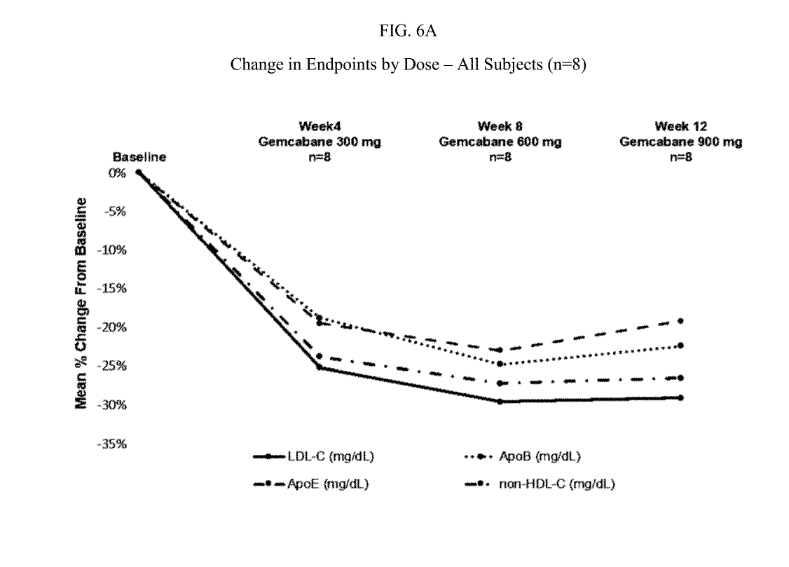

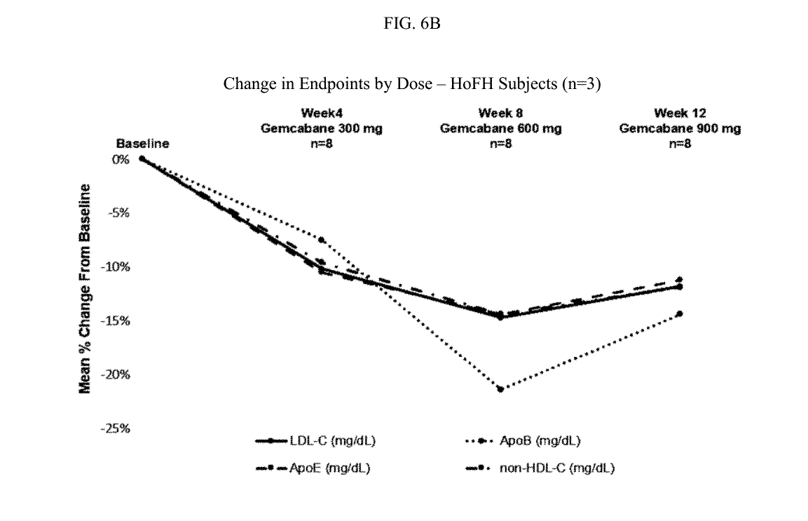

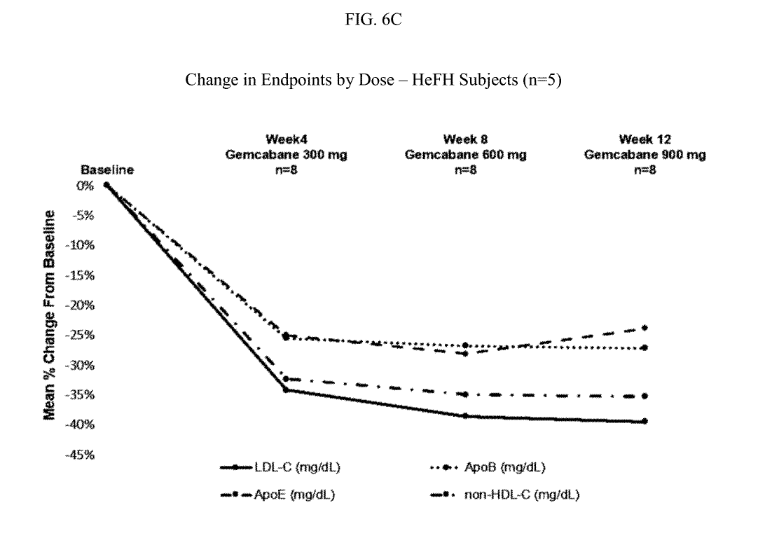

The patients were also analyzed for a number of secondary endpoints which are shown in Table 7 below. The percent from baseline numbers for LDL-C, Apo-E, ApoB, and non-HDL-C (all mg/dL) are shown in FIGS. 6A, 6B, and 6C.

TABLE 7
Mean Change in Secondary Endpoints by Dose

| | | Gemcabene 300 mg | | Gemcabene 600 mg | | Gemcabene 900 mg | |
|---|---|---|---|---|---|---|---|
| | | Week 4 | | Week 8 | | Week 12 | |
| | | | % | | % | | % |
| | | | Change | | Change | | Change |
| | | | from | | from | | from |
| | Baseline (SD) | Value (SD) | Baseline | Value (SD) | Baseline | Value (SD) | Baseline |
| LDL-C | 351.3 (181.8) | 275.6 (184.9) | −25.2% | 258.0 (180.4) | −29.6% | 262.9 (178.2) | −29.1% |
| (mg/dL) | | | | | | | |
| Total | 425.4 (167.1) | 339.4 (169.0) | −21.3% | 322.9 (166.0) | −24.6% | 327.0 (164.1) | −24.6% |
| Cholesterol | | | | | | | |
| (mg/dL) | | | | | | | |
| Non-HDL-C | 379.9 (177.3) | 298.8 (181.9) | −23.8% | 282.5 (177.0) | −27.2% | 287.1 (173.1) | −26.5% |
| (mg/dL) | | | | | | | |
| ApoB | 221.3 (97.3) | 181.9 (103.6) | −18.8% | 163.3 (80.7) | −24.8% | 171.8 (90.3) | −22.4% |
| (mg/dL) | | | | | | | |
| ApoE | 6.7 (1.7) | 5.3 (1.6) | −19.5% | 5.0 (1.8) | −23.0% | 5.23 (1.7) | −19.2% |
| (mg/dL) | | | | | | | |
| ApoC-III | 10.6 (4.1) | 9.3 (2.4) | −7.8% | 9.0 (2.07) | −9.7% | 9.3 (1.8) | −6.5% |
| (mg/dL) | | | | | | | |
| VLDL-C | 28.7 (12.5) | 23.1 (8.71) | −13.5% | 24.5 (7.5) | −8.4% | 24.3 (9.1) | −7.2% |
| (mg/dL) | | | | | | | |
| Triglycerides | 143.6 (62.7) | 116.4 (43.0) | −12.6% | 121.8 (37.3) | −9.01% | 121.3 (46.1) | −7.2% |
| (mg/dL) | | | | | | | |
| HDL-C | 45.4 (18.7) | 40.6 (18.5) | −11.89% | 40.4 (19.2) | −13.3% | 39.9 (19.3) | −12.9% |
| (mg/dL) | | | | | | | |
| Lp (a) | 113.5 (93.0) | 132.6 (127.3) | 6.3% | 131.8 (136.8) | 5.3% | 134.8 (132.0) | 15.0% |
| (mg/dL) | | | | | | | |
| hsCRP* | 3.75 | 5.2 | 38.7% | 2.5 | −33.3% | 2.05 | −45.3% |
| (mg/L) | | | | | | | |
| Fibrinogen | 436.9 (89.2) | 472.1 (110.3) | 8.5% | 403.4 (48.9) | −5.8% | 409.5 (55.03) | −4.8% |
| (mg/dL) | | | | | | | |
| Serum | 527.0 (182.9) | — | — | — | — | 598.2 (154.4) | 14.3% |
| PCSK9 | | | | | | | |
| (ng/mL) | | | | | | | |

*median, two patients had acute events that resolved by week 2

Safety and Tolerability of the treatments were monitored throughout the study. Table 8 shows the summary of the data.

TABLE 8

| Category | Total Reported |
|---|---|
| Any Treatment Emergent Adverse Events (TEAEs) | 27* |
| TEAEs possibly related to Gemcabene | 8 |
| TEAEs resulting in discontinuation of Gemcabene | 0 |
| Serious Adverse Events (SAEs) | 0 |
| Myopathies | 0 |
| Increase in ALT > 3 × ULN | 0 |
| Increase in total bilirubin > 3 × ULN | 0 |
| Increase in serum creatinine > 3 × ULN | 0 |
| Deaths | 0 |

*The majority of AEs were gastrointestinal. All AEs were mild intensity, except one unrelated AE that was moderate.

1. A method for treating a patient with familial hypercholesterolemia, the method comprising administering an effective dose of a compound, wherein the compound is 6-(5-carboxy-5-methyl-hexyloxy)-2,2-dimethyl-hexanoic acid, an ester thereof, a pharmaceutically acceptable salt thereof, or a hydrate thereof, to the patient with familial hypercholesterolemia, wherein the patient is on a lipid lowering therapy and is in need of further LDL-C lowering.

2-36. (canceled)

37. The method of claim 1, wherein the compound is 6-(5-carboxy-5-methyl-hexyloxy)-2,2-dimethyl-hexanoic acid and is administered as the monocalcium salt of 6-(5-carboxy-5-methyl-hexyloxy)-2,2-dimethyl-hexanoic acid.

38. The method of claim 1, wherein the patient has been on a stable dose of the lipid-lowering therapy for at least one week prior to the administration of the compound.

39. The method of claim 1, wherein the patient with familial hypercholesterolemia is clinically determined to have homozygous familial hypercholesterolemia.

40. The method of claim 1, wherein the patient with familial hypercholesterolemia is genetically confirmed to have homozygous familial hypercholesterolemia.

41. The method of claim 1, wherein the patient with familial hypercholesterolemia is genetically confirmed to have heterozygous familial hypercholesterolemia.

42. The method of claim 1, wherein the patient with familial hypercholesterolemia is genetically confirmed to have compound heterozygous familial hypercholesterolemia.

43. The method according to claim 1, wherein the patient is on one or more lipid-lowering therapy and has a LDL-C level≥70 mg/dL.

44. The method of claim 1, wherein the lipid-lowering therapy comprises administration of a cholesterol absorption inhibitor, a 3-hydroxy-3-methylglutaryl-coenzyme (HMG-CoA) reductase inhibitor, a proprotein converse subtilisin/kexin type 9 (PCSK9) inhibitor, an acetyl-CoA carboxylase (ACC) inhibitor, an ApoC-III inhibitor, an Apo E mimetic, an Apo B synthesis inhibitor, a microsomal triglyceride transfer protein inhibitor, an ATP-citrate lyase (ACL)-inhibitor, fish oil, eicosapentaenoic acid (EPA), Lovaza, an ethyl ester of eicosapentaenoic acid, docosahexaenoic acid, an ethyl ester of docosahexaenoic acid, nicotinic acid, bile acid sequestrant, a cholesteryl ester transfer protein (CETP) inhibitor or any combination thereof.

45. The method of claim 44, wherein the lipid-lowering therapy comprises administration of evolocumab, ezetimibe, mipomersen or lomitapide.

46. The method of claim 44, wherein the statin is atorvastatin, cerivastatin, rosuvastatin, simvastatin, pravastatin, lovastatin, fluvastatin, or pitavastatin.

47. The method of claim 1, wherein the effective dose of gemcabene is 25 mg/day, 50 mg/day, 75 mg/day, 150 mg/day, 300 mg/day, 450 mg/day, 600 mg/day, or 900 mg/day.

48. The method of claim 47, wherein the patient has a baseline LDL-C level and the baseline LDL-C level is reduced by at least 15%.

49. A method for reducing LDL-C in a patient with familial hypercholesterolemia, the method comprising administering an effective dose of a compound, wherein the compound is 6-(5-carboxy-5-methyl-hexyloxy)-2,2-dimethyl-hexanoic acid, an ester thereof, a pharmaceutically acceptable salt thereof, or a hydrate thereof, to the patient with familial hypercholesterolemia, wherein the patient is on a lipid lowering therapy and is in need of further LDL-C lowering.

50. The method of claim 49, wherein the compound is 6-(5-carboxy-5-methyl-hexyloxy)-2,2-dimethyl-hexanoic acid and is administered as the monocalcium salt of 6-(5-carboxy-5-methyl-hexyloxy)-2,2-dimethyl-hexanoic acid.

51. The method of claim 49, wherein the patient has been on a stable dose of the lipid-lowering therapy for at least one week prior to the administration of the compound.

52. The method of claim 49, wherein the patient with familial hypercholesterolemia is clinically determined to have homozygous familial hypercholesterolemia.

53. The method of claim 49, wherein the patient with familial hypercholesterolemia is genetically confirmed to have homozygous familial hypercholesterolemia.

54. The method of claim 49, wherein the patient with familial hypercholesterolemia is genetically confirmed to have heterozygous familial hypercholesterolemia.

55. The method of claim 49, wherein the patient with familial hypercholesterolemia is genetically confirmed to have compound heterozygous familial hypercholesterolemia.

56. The method according to claim 49, wherein the patient is on one or more lipid-lowering therapy and has a LDL-C level≥70 mg/dL.

57. The method of claim 49, wherein the lipid-lowering therapy comprises administration of a cholesterol absorption inhibitor, a HMG-CoA reductase inhibitor, a PCSK9 inhibitor, an ACC inhibitor, an ApoC-III inhibitor, an Apo E mimetic, an Apo B synthesis inhibitor, a microsomal triglyceride transfer protein inhibitor, an ACL-inhibitor, fish oil, EPA, Lovaza, an ethyl ester of eicosapentaenoic acid, docosahexaenoic acid, an ethyl ester of docosahexaenoic acid, nicotinic acid, bile acid sequestrant, a CETP inhibitor or any combination thereof.

58. The method of claim 57, wherein the lipid-lowering therapy comprises administration of evolocumab, ezetimibe, mipomersen or lomitapide.

59. The method of claim 57, wherein the statin is atorvastatin, cerivastatin, rosuvastatin, simvastatin, pravastatin, lovastatin, fluvastatin, or pitavastatin.

60. The method of claim 49, wherein the effective dose of gemcabene is 25 mg/day, 50 mg/day, 75 mg/day, 150 mg/day, 300 mg/day, 450 mg/day, 600 mg/day, or 900 mg/day.

61. The method of claim 60, wherein the patient has a baseline LDL-C level and the baseline LDL-C level is reduced by at least 15%.
